(19) United States
(12) Patent Application Publication
(10) Pub. No.: US 2010/0184818 A1
(43) Pub. Date: Jul. 22, 2010

(54) PHOTOSENSITIZERS FOR TARGETED PHOTODYNAMIC THERAPY

(75) Inventors: John Timothy Wharton, Bryan, TX (US); Hariprasad Gali, Edmond, OK (US); Michael R. Hamblin, Revere, MA (US); Pawel Mroz, Boston, MA (US)

Correspondence Address: EDWARDS ANGELL PALMER & DODGE LLP, P.O. BOX 55874, BOSTON, MA, 02205, US

(73) Assignees: THE GENERAL HOSPITAL CORPORATION, Boston, MA (US); LYNNTECH, INC., College Station, TX (US)

(21) Appl. No.: 12/598,007

(22) PCT Filed: Apr. 15, 2008
(86) PCT No.: PCT/US08/04865

§ 371 Date: Mar. 25, 2010

Related U.S. Application Data
(60) Provisional application No. 60/927,365, filed on May 2, 2007.

(51) Int. Cl. A61K 31/40; A61K 31/16; C12N 5/00; A61K 31/395; A61P 35/00

(52) U.S. Cl. 514/410; 514/616; 435/375; 514/183; 977/734

(57) ABSTRACT

The present invention provides photosensitizer compounds based on functionalized fullerenes useful in targeted photodynamic therapy (PDT), and methods of use thereof.

### RELATED APPLICATION

This application claims priority to U.S. provisional patent application Ser. No. 60/927,365, filed May 2, 2007, the entire content of which is incorporated herein by this reference.

### STATEMENT OF U.S. GOVERNMENT INTEREST

Funding for the present invention was provided in part by the Government of the United States under Grant Nos. R43CA103268, R44AI68400, R01CA/AI838801 and R01AI050875 from the National Institute of Health. Accordingly, the Government of the United States has certain rights in and to the invention.

### INCORPORATION BY REFERENCE

Each of the applications and patents cited in this text, as well as each document or reference cited in each of the applications and patents (including during the prosecution of each issued patent; “application cited documents”), and each of the PCT and foreign applications or patents corresponding to and/or paragraphing priority from any of these applications and patents, and each of the documents cited or referenced in each of the application cited documents, are hereby expressly incorporated herein by reference. More generally, documents or references are cited in this text, either in a Reference List, or in the text itself; and, each of these documents or references (“herein-cited references”), as well as each document or reference cited in each of the herein-cited references (including any manufacturer's specifications, instructions, etc.), is hereby expressly incorporated herein by reference.

### BACKGROUND OF THE INVENTION

### Photodynamic Therapy

Photodynamic therapy (PDT) refers to the use of photosensitizing drugs in combination with light for treating medical conditions. The PDT technique has shown promise as a cancer therapy (Dolmans, D. E., Fukumura, D., and Jain, R. K. (2003). Photodynamic therapy for cancer. Nat. Rev. Cancer 3, 380-387) and recently has achieved success as a treatment for age-related macular degeneration (Brown, S. B., and Mellish, K. J. (2001). Verteporfin: a milestone in ophthalmology and photodynamic therapy. Expert Opin. Pharmacother. 2, 351-361). The PDT method uses a compound known as a photosensitizer (PS) which is administered directly (e.g., endoscopically or topically) to an accessible treatment site, or alternatively, is administered systemically and concentrates in a target tissue site within the body of a subject. Subsequent irradiation of the target site with visible light of suitable wavelength generates singlet oxygen, irradiation of the target site with visible light of suitable wavelength generates singlet oxygen, ¹O2, within or on the surface of the cells of the treatment site, ultimately leading to cell death. The singlet oxygen is catalytically generated by energy transfer from the PS to oxygen from dissolved O₂, which is ubiquitous in the body's tissues.

Most PS used for photodynamic therapy (PDT) possess the tetrapyrrole backbone, and are at present used for medically approved applications in cancer therapy [8], ophthalmology [9] and dermatology [10]. In an analogous fashion to processes identified in traditional tetrapyrrole PS, illumination of fullerenes dissolved in organic solvents in the presence of oxygen, leads to the efficient generation of highly reactive singlet oxygen via energy transfer from the excited triplet state of the fullerene [11]. Recent reports have shown that in polar solvents, especially those containing reducing agents (such as NADH at concentrations found in cells), illumination will generate the reactive reduced oxygen species, superoxide anion (O₂*⁻) and hydroxyl radical [12, 13]. These two pathways are analogous to the Type II and Type I photochemical mechanisms frequently discussed in PDT with tetrapyrrole-based PS [14].

Photodynamic therapy is advantageous compared with other therapies due to its dual selectivity: not only is the PS targeted to the tumor or other lesion, but the light can also be accurately delivered to the affected tissue.

Fullerenes

Fullerenes (originally buckminsterfullerenes) are a class of carbon molecules; first discovered in 1985 [1]; which is composed of sixty carbon atoms arranged in a soccer-ball structure. The condensed aromatic rings present in the compound lead to an extended π-conjugated system of molecular orbitals and therefore to significant absorption of visible light. In recent years there has been much interest in studying possible biological activities of fullerenes (and other nanostructures produced in the nanotechnology revolution) with a view to using them in medicine [2-4]. An important issue when dealing with unmodified fullerenes is the absolute lack of solubility in polar or biologically compatible solvents for biological evaluation. Therefore fullerenes have to be chemically modified or functionalized in such a way that they acquire solubility and versatility [5-7].

The absorption of visible light referred to above allows fullerenes to act as photosensitizers (PS). Various fullerenes, including pristine C60 as well as functionalized derivatives, have been previously used to carry out in vitro PDT reactions leading to: cleavage of DNA strands [15-18], photoinactivation of viruses [19-21], production of oxidative damage to lipids in microsomal membranes [22, 23], PDT-induced killing of mammalian cells in tissue culture [7, 24-26] and even reports of regressions after PDT in a mouse tumor model [27, 28].

Given the urgent need for new cancer therapy agents and the PDT potential of fullerenes and functional derivatives of fullerene, it would be desirable to develop functionalized fullerene PS compounds that are effective for killing cancer cells by rapid induction of apoptosis after illumination, and that in contrast to many conventional PS, involve both Type I and Type II processes.

### SUMMARY OF THE INVENTION

The invention provides for the use of a new class of photosensitizing molecules for PDT for cancer. It has now been demonstrated that cationic fullerene embodiments functionalized with one, two, or three pyrrolidinium groups, after a short incubation followed by illumination with white light, have a broad-spectrum antitumor activity and can rapidly induce apoptosis and tumor cell death.

In this invention, fullerene molecules, e.g., C₆₀, C₇₀, C₇₄, C₇₆, C₇₈, C₈₀, C₈₂, C₈₄, higher fullerenes and their functionalized derivatives, have been modified to include a variety of properties needed for application of PDT to cancer therapy. This was achieved by controlling hydrophobicity, molecular charge, and water solubility of the carbon nanomaterial specifically to target tumor cells preferentially over other types of cells for PDT. A positive charge on some embodiments allows the fullerenes to selectively bind to certain tumor cells. Monocationic fullerenes in particular perform well as cancer therapy photosensitizers resulting in rapid induction of apoptosis after illumination. Accordingly, cationic fullerene-mediated photodynamic therapy may find significant application in cancer treatment.

More particularly, in one embodiment the present invention provides compositions comprising a functionalized fullerene, wherein the wherein the functionalized fullerene comprises a fullerene core (Cₙ) where n is an even integer greater than or equal to 60, and at least one functional group bonded to at least one carbon atom of the fullerene core.

Some embodiments are based on hydrophilic cationic fullerene derivatives. Other embodiments are hydrophilic neutral fullerene derivatives.

Fullerene derivatives of the invention are suitable for the treatment of a variety of cancers and tumors. Accordingly, in another embodiment, the invention provides a method for providing cancer therapy, which includes administering an effective amount of a functionalized fullerene species to a subject in need thereof. The fullerene species can be any one of the compounds described herein. The method includes directing light onto the administered fullerene species to produce a cytotoxic species; and killing cancer cells associated with or proximal to the fullerene species by reaction with the cytotoxic species.

### BRIEF DESCRIPTION OF THE DRAWINGS

The following Detailed Description, given by way of example, but not intended to limit the invention to specific embodiments described, may be understood in conjunction with the accompanying drawings, incorporated herein by reference. Various preferred features and embodiments of the present invention will now be described by way of non-limiting examples and with reference to the accompanying drawings in which:

FIG. 1 shows the structures of six functionalized fullerenes useful in the treatment of cancer and hyperproliferative diseases. (A) BF1-BF3, (B) BF4-BF6.

FIG. 2 is a graph showing UV-visible absorption spectra of (A) BF1-BF3 and (B) BF4-BF6 in DMSO:water 1:9.

FIG. 3 is a graph showing the MMT assay survival curves of (A) LLC; (B) J774; and (C) CT26 cells after 24 h incubation with 2 μM BF1-BF6 followed by a wash and illumination with white light. The Values are means of 9 separate wells and bars are SD.

FIG. 4 shows Fluorescence micrographs of J774 cells that had been incubated with the intracellular ROS probe H2DCFDA, illuminated with 5 J/cm2 405 nm laser and imaged after 5 mM. (A) H2DCFDA without fullerene; (B) BF4 for 24 hours+H2DCFDA. Scale bar is 100 μum.

FIG. 5 is a graph showing the time course of apoptosis as measured by a fluorescent caspase assay in CT26 cells receiving BF4-PDT (80% lethal dose) or BF6-PDT 60% lethal dose).

FIG. 6 shows three graphs showing the time decay curves of 1270-nm luminescence from singlet oxygen produced when BF4 (49 μM), BF6 (52 μM) or riboflavin (RBFL, 17 μM) were excited with a 5-ns 449-nm laser pulse. (A) deuterated methanol; (B) deuterated PBS; (C) compare BF6 in air or in nitrogen.

FIG. 7 is a graph showing the increase with illumination time (broad band white light) in ESR signal from superoxide-specific spin trap (DMPO—OOH) and BF4 or BF6 (35 μM) in presence of 1 mM NADH or 2 mM histidine in 1:3 H₂O:DMSO.

FIG. 8 is a chart showing Oxygen consumption rates for BF4 or BF6 (35 μM) in presence of 1 mM NADH or 2 mM histidine with or without 5-mM sodium azide in 1:3H2O:DMSO determined by ESR oximetry.

### DETAILED DESCRIPTION OF THE INVENTION

### I. Definitions

In order that the invention may be more readily understood, certain terms are first defined and collected here for convenience. Other definitions appear in context throughout the application.

The term “alkyl” refers to the radical of saturated aliphatic groups, including straight-chain alkyl groups, branched-chain alkyl groups, cycloalkyl (alicyclic) groups, alkyl substituted cycloalkyl groups, and cycloalkyl substituted alkyl groups. The term alkyl further includes alkyl groups, which can further include oxygen, nitrogen, sulfur or phosphorous atoms replacing one or more carbons of the hydrocarbon backbone. In certain embodiments, a straight chain or branched chain alkyl has 30 or fewer carbon atoms in its backbone (e.g., C₁-C₃₀ for straight chain, C₃-C₃₀ for branched chain), preferably 26 or fewer, and more preferably 20 or fewer. Likewise, certain cycloalkyls have from 3-10 carbon atoms in their ring structure, and more preferably have 3, 4, 5, 6 or 7 carbons in the ring structure.

Moreover, the term alkyl as used throughout the specification and claims is intended to include both “unsubstituted alkyls” and “substituted alkyls,” the latter of which refers to alkyl moieties having substituents replacing a hydrogen on one or more carbons of the hydrocarbon backbone. Such substituents can include, for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, trifluoromethyl, cyano, azido, heterocyclyl, alkylaryl, or an aromatic or heteroaromatic moiety. It will be understood by those skilled in the art that the moieties substituted on the hydrocarbon chain can themselves be substituted, if appropriate. Cycloalkyls can be further substituted, e.g., with the substituents described above. An “alkylaryl” moiety is an alkyl substituted with an aryl (e.g., phenylmethyl(benzyl)). The term “alkyl” also includes unsaturated aliphatic groups analogous in length and possible substitution to the alkyls described above, but that contain at least one double or triple bond respectively.

Unless the number of carbons is otherwise specified, “lower alkyl” as used herein means an alkyl group, as defined above, but having from one to ten carbons, more preferably from one to six, and most preferably from one to four carbon atoms in its backbone structure, which may be straight or branched-chain.

The terms “alkoxyalkyl,” “polyaminoalkyl” and “thioalkoxyalkyl” refer to alkyl groups, as described above, which further include oxygen, nitrogen or sulfur atoms replacing one or more carbons of the hydrocarbon backbone, e.g., oxygen, nitrogen or sulfur atoms.

The terms “alkenyl” and “alkynyl” refer to unsaturated aliphatic groups analogous in length and possible substitution to the alkyls described above, but that contain at least one double or triple bond, respectively.

The term “aryl” as used herein, refers to the radical of aryl groups, including 5- and 6-membered single-ring aromatic groups that may include from zero to four heteroatoms, for example, benzene, pyrrole, furan, thiophene, imidazole, benzoxazole, benzothiazole, triazole, tetrazole, pyrazole, pyridine, pyrazine, pyridazine and pyrimidine, and the like. Aryl groups also include polycyclic fused aromatic groups such as naphthyl, quinolyl, indolyl, and the like.

Those aryl groups having heteroatoms in the ring structure may also be referred to as “aryl heterocycles,” “heteroaryls” or “heteroaromatics.” The aromatic ring can be substituted at one or more ring positions with such substituents as described above, as for example, halogen, hydroxyl, alkoxy, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, trifluoromethyl, cyano, azido, heterocyclyl, alkylaryl, or an aromatic or heteroaromatic moiety. Aryl groups can also be fused or bridged with alicyclic or heterocyclic rings which are not aromatic so as to form a polycycle (e.g., tetralin).

The term “chiral” refers to molecules which have the property of non-superimposability of the mirror image partner, while the term “achiral” refers to molecules which are superimposable on their mirror image partner.

The term “enantiomers” refers to two stereoisomers of a compound which are non-superimposable mirror images of one another. An equimolar mixture of two enantiomers is called a “racemic mixture” or a “racemate.”

The term “halogen” designates —F, —Cl, —Br or —I.

The term “haloalkyl” is intended to include alkyl groups as defined above that are mono-, di- or polysubstituted by halogen, e.g., fluoromethyl and trifluoromethyl.

The term “hydroxyl” means —OH.

The term “heteroatom” as used herein means an atom of any element other than carbon or hydrogen. Preferred heteroatoms are nitrogen, oxygen, sulfur and phosphorus.

The term “isomers” or “stereoisomers” refers to compounds which have identical chemical constitution, but differ with regard to the arrangement of the atoms or groups in space. Furthermore the indication of stereochemistry across a carbon-carbon double bond is also opposite from the general chemical field in that “Z” refers to what is often referred to as a “cis” (same side) conformation whereas “E” refers to what is often referred to as a “trans” (opposite side) conformation. With respect to the nomenclature of a chiral center, the terms “d” and “l” configuration are as defined by the IUPAC Recommendations. As to the use of the terms, diastereomer, racemate, epimer and enantiomer, these will be used in their normal context to describe the stereochemistry of preparations.

The term “obtaining” as in “obtaining the fullerene derivative” is intended to include purchasing, synthesizing or otherwise acquiring the fullerene derivative (or indicated substance or material).

A “photosensitizer” or “photosensitive material” is defined herein as a material, element, chemical, solution, compound, matter, or substance which is sensitive, reactive, receptive, or responsive to light energy. The term can refer to a photoactivatable fullerene compound, or a precursor thereof, that produces a reactive species (e.g., oxygen) having a phototoxic effect on a tumor cell.

The terms “polycyclyl” or “polycyclic radical” refer to the radical of two or more cyclic rings (e.g., cycloalkyls, cycloalkenyls, cycloalkynyls, aryls and/or heterocyclyls) in which two or more carbons are common to two adjoining rings, e.g., the rings are “fused rings”. Rings that are joined through non-adjacent atoms are termed “bridged” rings. Each of the rings of the polycycle can be substituted with such substituents as described above, as for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, trifluoromethyl, cyano, azido, heterocyclyl, alkyl, alkylaryl, or an aromatic or heteroaromatic moiety.

The term “sulfhydryl” or “thiol” means —SH.

The term “subject” refers to animals such as mammals, including, but not limited to, primates (e.g., humans), cows, sheep, goats, horses, dogs, cats, rabbits, rats, mice and the like. In certain embodiments, the subject is a human.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited are not changed by the presence of more than that which is recited, but excludes prior art embodiments.

Unless otherwise defined, all technical and scientific terms used herein have the same meaning as commonly understood by one of ordinary skill in the art to which this invention belongs. In case of conflict, the present application, including definitions will control.

### II. Compositions of the Invention

The present invention provides photodynamic compositions for PDT. PDT employs photoactivatable compounds known as photosensitizers to selectively target and destroy cells. Therapy involves delivering visible light of the appropriate wavelength to excite the photosensitizer molecule to the excited singlet state. This excited state can then undergo intersystem crossing to the slightly lower energy triplet state, which can then react further by one or both of two pathways, known as Type I and Type II photoprocesses (Ochsner (1997) J Photochem Photobiol B 39:1-18). The Type I pathway involves electron transfer reactions from the photosensitizer triplet to produce radical ions that can then react with oxygen to produce cytotoxic species such as superoxide, hydroxyl and lipid derived radicals. The Type II pathway involves energy transfer from the photosensitizer triplet to ground state molecular oxygen (triplet) to produce the excited state singlet oxygen, which can then oxidize many biological molecules such as proteins, nucleic acids and lipids, and lead to cytotoxicity.

Functionalized Fullerenes as Photosensitizers

The therapeutic compositions of the invention comprise novel photosensitizer compounds for PDT based on functionalized fullerene molecules. Without being bound by theory, it is believed that the functionalized fullerene molecules of the invention function through both the Type I and Type II pathway described herein above.

More particularly, the invention provides fullerenes, e.g., C₆₀, C₇₀, C₇₄, C₇₆, C₇₈, C₈₀, C₈₂, C₈₄, higher fullerenes and their functionalized derivatives. Buckminsterfullerenes, also known as fullerenes or, more colloquially, “buckyballs,” are cage-like molecules consisting essentially of sp²-hybridized carbons. Fullerenes were first reported by Kroto et al., Nature (1985) 318:162. Fullerenes are the third form of pure carbon, in addition to diamond and graphite. Typically, fullerenes are arranged in hexagons, pentagons, or both. Most known fullerenes have 12 pentagons and varying numbers of hexagons depending on the size of the molecule. Common fullerenes include C₆₀ and C₇₀, although fullerenes comprising up to about 400 carbon atoms are also known. Exemplary functionalized fullerenes are described in WO2006/093891

C₆₀ has 30 carbon-carbon double bonds, and has been reported to readily react with oxygen radicals (Krusic et al., Science, 1991, 254:1183-1185). Other fullerenes have comparable numbers of carbon-carbon double bonds and would be expected to be similarly reactive with oxygen radicals. Native fullerenes are generally only soluble in apolar organic solvents, such as toluene or benzene. To render fullerenes water-soluble, as well as to impart other properties to fullerene-based molecules, a number of fullerene substituents have been developed.

Methods of substituting fullerenes with various substituents are known in the art. Methods include 1,3-dipolar additions (Sijbesma et al., J. Am. Chem. Soc. (1993) 115:6510-6512; Suzuki, J. Am. Chem. Soc. (1992) 114:7301-7302; Suzuki et al., Science (1991) 254:1186-1188; Prato et al., J. Org. Chem. (1993) 58:5578-5580; Vasella et al., Angew. Chem. Int. Ed. Engl. (1992) 31:1388-1390; Prato et al., J. Am. Chem. Soc. (1993) 115:1148-1150; Maggini et al., Tetrahedron Lett. (1994) 35:2985-2988; Maggini et al., J. Am. Chem. Soc. (1993) 115:9798-9799; and Meier et al., J. Am. Chem. Soc. (1994) 116:7044-7048), Diels-Alder reactions (lyoda et al., J. Chem. Soc. Chem. Commun. (1994) 1929-1930; Belik et al., Angew. Chem. Int. Ed. Engl. (1993) 32:78-80; Bidell et al., J. Chem. Soc. Chem. Commun. (1994) 1641-1642; and Meidine et al., J. Chem. Soc. Chem. Commun. (1993) 1342-1344), other cycloaddition processes (Saunders et al., Tetrahedron Lett. (1994) 35:3869-3872; Tadeshita et al., J. Chem. Soc. Perkin. Trans. (1994) 1433-1437; Beer et al., Angew. Chem. Int. Ed. Engl. (1994)33:1087-1088; Kusukawa et al., Organometallics (1994) 13:4186-4188; Averdung et al., Chem. Ber. (1994) 127:787-789; Akasaka et al., J. Am. Chem. Soc. (1994) 116:2627-2628; Wu et al., Tetrahedron Lett. (1994) 35:919-922; and Wilson, J. Org. Chem. (1993) 58:6548-6549); cyclopropanation by addition/elimination (Hirsch et al., Agnew. Chem. Int. Ed. Engl. (1994) 33:437-438 and Bestmann et al., C. Tetra. Lett. (1994) 35:9017-9020); and addition of carbanions/alkyl lithiums/Grignard reagents (Nagashima et al., J. Org. Chem. (1994) 59:1246-1248; Fagan et al., J. Am. Chem. Soc. (1994) 114:9697-9699; Hirsch et al., Agnew. Chem. Int. Ed. Engl. (1992) 31:766-768; and Komatsu et al., J. Org. Chem. (1994) 59:6101-6102); among others. The synthesis of substituted fullerenes is reviewed by Murphy et al., U.S. Pat. No. 6,162,926.

The discovery of the fullerenes in 1985, and the subsequent development of synthetic methods for the preparation of large-scale quantities of the allotropes of carbon has generated considerable interest and opened a whole new field of carbon chemistry. Fullerenes are defined as closed-cage polyhedrons made up entirely of sp²-hybridized carbon atoms that contain exactly 12 pentagonal faces (known as Euler's theorem) and (n/2−10) hexagonal faces where n is the number of carbon atoms (n must be even and greater than twenty). The soccer ball-shaped fullerene C₆₀ has the highest theoretically possible symmetry, icosahedral (Iₕ). It is the most abundant fullerene that is produced during the graphite combustion production of the materials, followed by C₇₀.

C₆₀ can be functionalized by well known methods of synthetic organic chemistry. The formation of C₆₀ derivatives (i.e., covalently modified C₆₀) nearly always involves the addition of a functional group (addend) across one or more of its 30 double bonds. When only one addend is attached, the fullerene derivative is termed a “monoadduct,” with two, a “bisadduct,” etc.

Another advantage of the spherical C₆₀ molecule for PDT is its large surface area of ˜200 Å compared to ≦100 Å² for other “flat” rigid PS, maximizing exposure to O₂. Additionally, the versatility of the C₆₀ scaffolding allows a tailoring of the hydrophobicity/hydrophilicity by simple synthetic methods, providing, as a nonlimiting example, any of a number of structures expected to be absorbed through the skin. Advantageously, C₆₀ and its derivatives are also thermally and photochemically stable (minimal photobleaching).

The present invention, in one aspect, provides compositions comprising a functionalized (substituted, derivatized) fullerene comprising a fullerene core (Cₙ) where n is an even integer greater than or equal to 60, and at least one functional group bonded to at least one carbon atom of the fullerene core.

In one embodiment, the functionalized fullerene is a compound of the generic formula I:

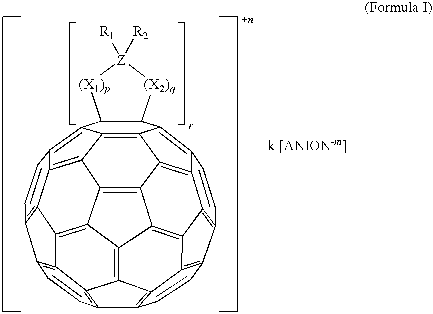

wherein

Z is carbon, nitrogen or phosphorus;

R₁ and R₂ are independently selected from the group consisting of C₁-C₁₂alkyl, C₂-C₁₂alkenyl, C₂-C₁₂alkynyl, C₃-C₈cycloalkyl, (aryl)C₀-C₄alkyl, (heteroaryl)C₀-C₄alkyl, or a group of the formula C(O)—N(R₄)(R₅)(R₆); or

ZR₁R₂ taken in combination form a 3-20 member heterocyclic ring having 1-6 ring heteroatoms selected from nitrogen and phosphorus and having at least one quaternary ammonium cation or quaternary phosphonium cation;

R₄ and R₅ are independently selected from hydrogen or a group selected from C₁-C₁₂alkyl, C₂-C₁₂alkenyl, C₂-C₁₂alkynyl, C₃-C₇cycloalkyl, C₃-C₈ (aryl)C₀-C₄alkyl, and (heteroaryl)C₀-C₄alkyl each of which groups is substituted with 0-3 substituents selected from hydroxy, amino, mono-, di-, or tri-(C₁-C₄alkyl)amino, halogen, quaternary ammonium cations, quaternary phosphonium cations;

R₆ is absent, hydrogen or a group selected from C₁-C₁₂alkyl, C₂-C₁₂alkenyl, C₂-C₁₂alkynyl, C₃-C₇cycloalkyl, C₃-C₈cycloalkyl, (aryl)C₀-C₄alkyl, and (heteroaryl)C₀-C₄alkyl each of which groups is substituted with 0-3 substituents selected from hydroxy, amino, mono-, di-, or tri-(C₁-C₄alkyl)amino, halogen, quaternary ammonium cations, quaternary phosphonium cations;

X₁ and X₂ are independently selected at each occurrence from the group consisting of CH₂ and CHR₃, wherein R₃ is a C₁-C₆alkyl which is independently selected at each occurrence of R₃;

r is 1, 2, 3, or 4;

p and q are independently selected from 0, 1, 2, or 3 such that 0≦(p+q)≦4;

ANION is at least one organic or inorganic anion;

m is a negative integer corresponding to the net negative charge of each ANION equivalent;

n is a positive integer corresponding to the net positive charge of the substituted buckminsterfullerene cation; and

k is the quotient of n/m.

Certain other compounds of formula I include those compounds in which the C60-fullerene is substituted by a Cn-fullerene wherein n is an integer of between 50 and about 84.

Another embodiment is a compound according to formula I, wherein

Z is nitrogen or phosphorus;

X₁ and X₂ are methylene;

p=q=1;

R₁ and R₂ are independently selected C₁-C₆alkyl, (aryl)C₀-C₁alkyl, or (heteroaryl)C₀-C₁alkyl;

r is 2, 3, or 4; and

n≧r.

Another embodiment is a compound according to formula I, referred to herein as compounds of formula II, wherein

Z is nitrogen or phosphorus;

X₁ and X₂ are methylene;

p=q=1;

R₁ is C₁-C₆alkyl, (aryl)C₀-C₁alkyl, or (heteroaryl)C₀-C₁alkyl;

R₂ is (aryl)C₀-C₁alkyl, or (heteroaryl)C₀-C₁alkyl;

r is 1, 2, 3, or 4; and

n≧r.

Another embodiment is a compound according to formula II, wherein

Z is nitrogen;

X₁ and X₂ are methylene;

p=q=1;

R₁ and R₂ are independently selected from methyl, ethyl, propyl or isopropyl;

r is 2, 3, or 4; and

n≧r

Another embodiment is the compound according to formula I, wherein

Z is carbon;

p=q=0;

R₁ and R₂ are independently selected groups of the formula C(O)—N(R₄)(R₅)(R₆); or

ZR₁R₂ taken in combination form a 6-20 member heterocyclic ring having 1-6 ring heteroatoms selected from nitrogen and phosphorus and having at least one quaternary ammonium cation or quaternary phosphonium cation;

R₄ and R₅ are independently selected from hydrogen or a group selected from C₁-C₁₂alkyl, C₂-C₁₂alkenyl, C₂-C₁₂alkynyl, C₃-C₇cycloalkyl, C₃-C₈ (aryl)C₀-C₄alkyl, and (heteroaryl)C₀-C₄alkyl each of which groups is substituted with 0-3 substituents selected from hydroxy, amino, di-, or tri-(C₁-C₂alkyl)amino, halogen, quaternary ammonium cations, quaternary phosphonium cations; and

R₆ is absent, hydrogen or a group selected from C₁-C₁₂alkyl, C₂-C₁₂alkenyl, C₂-C₁₂alkynyl, C₃-C₇cycloalkyl, C₃-C₈cycloalkyl, (aryl)C₀-C₄alkyl, and (heteroaryl)C₀-C₄alkyl each of which groups is substituted with 0-3 substituents selected from hydroxy, amino, mono-, di-, or tri-(C₁-C₂alkyl)amino, halogen, quaternary ammonium cations, quaternary phosphonium cations.

Another embodiment is a compound according to formula II, referred to herein as formula III, wherein

R₁ and R₂ are independently selected groups of the formula C(O)—N(R₄)(R₅)(R₆);

R₄ is C₂-C₆alkyl substituted with 1-3 substitutents selected from hydroxy, amino, di-, or tri-(C₁-C₂alkyl)amino, and quaternary ammonium cations;

R₅ is hydrogen, C₁-C₆alkyl substituted with 0-3 substitutents selected from hydroxy, amino, and quaternary ammonium cations; and

R₆ is absent, hydrogen, or C₁-C₆alkyl substituted with 0-3 substitutents selected from hydroxy, amino, di-, or tri-(C₁-C₂alkyl)amino, and quaternary ammonium cations.

Another embodiment is a compound according to formula III, referred to herein as formula IV, R₁ and R₂ are the same and are selected from the group consisting of:

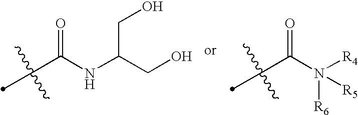

wherein R₄ is methyl, ethyl or propyl or isopropyl;

R₅ and R₆ are independently selected from methyl, ethyl, 2-(N,N-dimethylamino)ethyl, 3-(N,N-dimethylamino)propyl, 2-(N,N,N-trimethylammonium)ethyl, or 3-(N,N,N-trimethylammonium)propyl.

Another embodiment is a compound according to formula IV, wherein r is 1.

Another embodiment is a compound according to formula IV, wherein r is 2.

Another embodiment is a compound according to formula IV, wherein r is 3.

Another embodiment is a compound according to formula I, wherein

p=q=0; and

ZR₁R₂, taken in combination, form a 7-20 member heterocyclic ring having 2 to 6 nitrogen atoms wherein at least one of the nitrogen atoms is a quaternary ammonium cation. (Formula V).

Another embodiment is a compound according to formula V, referred to herein as formula VI wherein ZR₁R₂ is a heterocyclic ring of the formula:

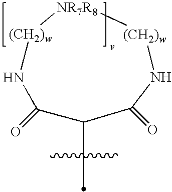

wherein

w is independently selected at each occurrence from 1, 2 or 3;

v is 0, 1, 2, or 3;

R₇ is independently selected at each occurrence from hydrogen, C₁-C₆alkyl substituted with 0-3 substitutents selected from hydroxy, amino, and quaternary ammonium cations; and

R₈ is independently selected at each occurrence from absent, hydrogen, or C₁-C₆alkyl substituted with 0-3 substitutents selected from hydroxy, amino, di-, or tri-(C₁-C₂alkyl)amino, and quaternary ammonium cations; and wherein at least one NR₇R₈ is a quaternary ammonium cation or is substituted by a quaternary ammonium cation.

Another embodiment is a compound according to formula VI, wherein

v is 1, 2 or 3;

w is 2;

R₇ is independently selected from the group of methyl, ethyl or propyl or isopropyl;

R₈ are independently selected from methyl, ethyl, 2-(N,N-dimethylamino)ethyl, 3-(N,N-dimethylamino)propyl, 2-(N,N,N-trimethylammonium)ethyl, or 3-(N,N,N-trimethylammonium)propyl.

The chemical structures of certain preferred embodiments of the fullerene-based photosensitizer compounds of the invention are shown in Table 1.

TABLE 1
Chemical structures of the fullerene derivatives. 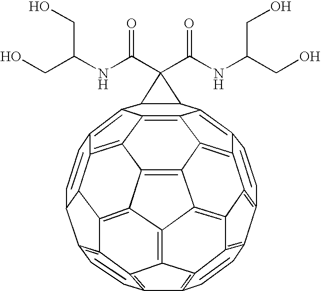
NI1 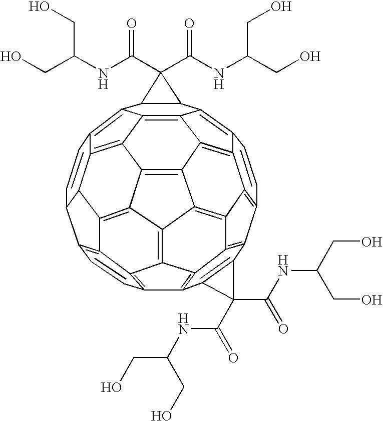
NI2 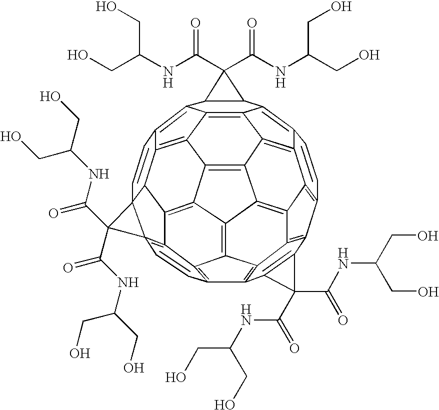
NI3 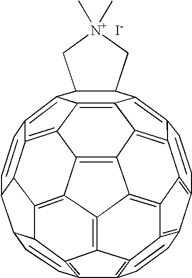
CI1 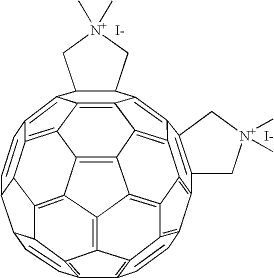
CI2 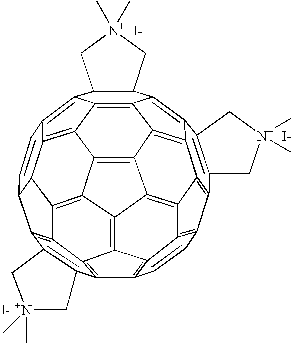
CI3 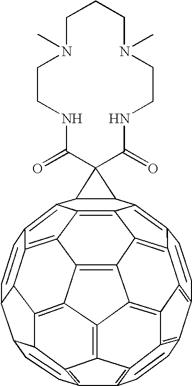
N1
NI = non-ionic, CI = cationic, N = nitrogenous base.

Synthetic schemes for particular functionalized fullerene PS are further described in Examples 1-4, infra.

A pharmaceutical composition in accordance with the invention can contain a suitable concentration of an active agent (i.e., a functionalized fullerene compound) and may also comprise certain other components. For example, in some embodiments, pharmaceutical compositions of the present invention are formulated with pharmaceutically acceptable carriers or excipients, such as water, saline, aqueous dextrose, glycerol, or ethanol, and may also contain auxiliary substances such as wetting or emulsifying agents, and pH buffering agents in addition to the active agent.

The pharmaceutical composition can also comprise, or can be applied in combination with, an optical clearing agent to enhance the photoactive effectiveness of the functionalized fullerene compound by allowing more effective penetration of light through tissue. At visible and near infrared wavelengths, optical scattering dominates over absorption and is much more significant in reducing light penetration into biological tissues. Optical clearing is a method for inducing a transient reduction in optical scattering by biological tissue. Studies have demonstrated increased light penetration depth using hyperosmotically active chemical agents such as glycerol, propylene glycol, ethylene glycol, DMSO, glucose or dextrose, oleic acid, linoleic acid, etc., which are applied to the skin or tissue. Various mechanisms for optical clearing have been proposed. Although the mechanism of optical clearing is still not entirely understood, it has been inferred that hyperosmotic agents reduce random scattering primarily by better refractive index matching, dehydration, and collagen dissociation.

One or more optical clearing agents can be applied to tissue simultaneously with the pharmaceutical composition, as a combined formulation. Alternatively, one or more optical clearing agents can be applied some time prior to the application of the pharmaceutical composition, as a separate formulation. One or more optical clearing agents can be applied to tissue simultaneously with the application of light or can be applied some time prior to the application of light.

The pharmaceutical composition can further comprise or be used in combination with a permeation enhancer (also termed an “absorption enhancer”), which promotes the distribution and penetration of the functionalized fullerene compound in the tissue being treated by PDT. Examples include but are not be limited to: DMSO, polyethylene glycol, nonionic surfactants, ionic surfactants, vitamin A, and steroids.

Kits

The invention also includes kits for inducing apoptosis and/or treating tumors in a subject comprising a functionalized fullerene compound and instructions for using the functionalized fullerene compound to treat the cancer in accordance with the methods described herein.

The kits of the invention include instructions for the reagents, equipment (test tubes, reaction vessels, needles, syringes, etc.), standards for calibrating the administration, and/or equipment provided or used to conduct the treatment. The standard or control information can be compared to a test sample to determine, for example, if the dosage is correct.

### III. Methods of the Invention

Cancer and Hyper-Proliferative Disorders

Photodynamic therapy according to the present invention may be utilized in the treatment of mammalian hyper-proliferative disorders. PDT can be utilized to inhibit, block, reduce, decrease, etc., cell proliferation and/or cell division, and/or produce apoptosis. This method administering to a mammal in need thereof, including a human, an amount of a functionalized fullerene of this invention, or a pharmaceutically acceptable salt, isomer, polymorph, metabolite, hydrate, solvate or ester thereof; directing light onto the administered fullerene compound to produce a cytotoxic species; and inhibiting, blocking, reducing, decreasing, etc., cell proliferation and/or cell division, and/or inducing apoptosis in cells associated with or proximal to the fullerene compound by reaction with the cytotoxic species, thereby providing anti-hyperproliferative therapy. Hyper-proliferative disorders include but are not limited, e.g., psoriasis, keloids, and other hyperplasias affecting the skin, benign prostate hyperplasia (BPH), solid tumors, such as cancers of the breast, respiratory tract, brain, reproductive organs, digestive tract, urinary tract, eye, liver, skin, head and neck, thyroid, parathyroid and their distant metastases. Those disorders also include lymphomas, sarcomas, and leukemias.

Examples of breast cancer include, but are not limited to invasive ductal carcinoma, invasive lobular carcinoma, ductal carcinoma in situ, and lobular carcinoma in situ.

Examples of cancers of the respiratory tract include, but are not limited to small-cell and non-small-cell lung carcinoma, as well as bronchial adenoma and pleuropulmonary blastoma.

Examples of brain cancers include, but are not limited to brain stem and hypothalamic glioma, cerebellar and cerebral astrocytoma, medulloblastoma, ependymoma, as well as neuroectodermal and pineal tumor.

Tumors of the male reproductive organs include, but are not limited to prostate and testicular cancer. Tumors of the female reproductive organs include, but are not limited to endometrial, cervical, ovarian, vaginal; and vulvar cancer, as well as sarcoma of the uterus.

Tumors of the digestive tract include, but are not limited to anal, colon, colorectal, esophageal, gallbladder, gastric, pancreatic, rectal, small-intestine, and salivary gland cancers.

Tumors of the urinary tract include, but are not limited to bladder, penile, kidney, renal pelvis, ureter, urethral and human papillary renal cancers.

Eye cancers include, but are not limited to intraocular melanoma and retinoblastoma.

Examples of liver cancers include, but are not limited to hepatocellular carcinoma (liver cell carcinomas with or without fibrolamellar variant), cholangiocarcinoma (intrahepatic bile duct carcinoma), and mixed hepatocellular cholangiocarcinoma.

Skin cancers include, but are not limited to squamous cell carcinoma, Kaposi's sarcoma, malignant melanoma, Merkel cell skin cancer, and non-melanoma skin cancer.

Head-and-neck cancers include, but are not limited to laryngeal, hypopharyngeal, nasopharyngeal, oropharyngeal cancer, lip and oral cavity cancer and squamous cell. Lymphomas include, but are not limited to AIDS-related lymphoma, non-Hodgkin's lymphoma, cutaneous T-cell lymphoma, Burkitt lymphoma, Hodgkin's disease, and lymphoma of the central nervous system.

Sarcomas include, but are not limited to sarcoma of the soft tissue, osteosarcoma, malignant fibrous histiocytoma, lymphosarcoma, and rhabdomyosarcoma.

Leukemias include, but are not limited to acute myeloid leukemia, acute lymphoblastic leukemia, chronic lymphocytic leukemia, chronic myelogenous leukemia, and hairy cell leukemia.

These disorders have been well characterized in humans, but also exist with a similar etiology in other mammals, and can be treated by administering pharmaceutical compositions of the present invention.

The term “treating” or “treatment” as stated throughout this document is used conventionally, e.g., the management or care of a subject for the purpose of combating, alleviating, reducing, relieving, improving the condition of, etc., of a disease or disorder, such as a carcinoma.

Administration

An “effective amount” of a functionalized fullerene compound is an amount sufficient to effect a beneficial or desired clinical result (e.g., a photodynamic effect). An effective amount can be administered in one or more doses. In terms of treatment, an effective amount is an amount that is sufficient to palliate, ameliorate, stabilize, reverse or slow the progression of a condition caused by infection. The effective amount is generally determined by the physician on a case-by-case basis and is within the skill of one in the art. In accordance with certain preferred aspects of the invention, “an effective amount of a functionalized fullerene compound” of the invention is defined as an amount sufficient to yield an acceptable anticancer effect, i.e., to kill tumor cells or to induce apoptosis in the mammalian subject of the PDT treatment.

As a rule, the dosage for in vivo therapeutics will vary. Several factors are typically taken into account when determining an appropriate dosage. These factors include age, sex and weight of the patient, the condition being treated, and the severity of the condition.

Suitable dosages and formulations of functionalized fullerene compound can be empirically determined by the administering physician. Standard texts, such as Remington: The Science and Practice of Pharmacy, 17th edition, Mack Publishing Company, and the Physician's Desk Reference, each of which is incorporated herein by reference, can be consulted to prepare suitable compositions and doses for administration. A determination of the appropriate dosage is within the skill of one in the art given the parameters for use described herein.

Administration can be in any order. Typically the functionalized fullerene compound is administered, followed by application of light. A light source is utilized to practice embodiments of the present invention. The light source may be laser light source, a high intensity flash lamp, a light-emitting diode (LED) or other illumination source as appreciated by those skilled in the relevant arts. A broad-spectrum light source may be utilized; however a narrow spectrum light source is one preferred light source. The light source may be selected with reference to the specific photosensitive material, as photosensitive materials may have an associated range of photoactivation. In some instances a laser light source may be used to practice the present invention. A variety of laser light sources is currently available, and the selection of a particular laser light source for implementing the PDT would readily be appreciated by those skilled in the relevant arts. A laser source may be selected with regard to the choice of wavelength, beam diameter, exposure time and sensitivity of the cellular and/or acellular organisms.

In preferred embodiments, the light source is utilized for a period of time necessary to effect a photodynamic response. The period of time for photodynamic activation of the photosensitive material is preferably between 5 seconds and 1 hour. Even more preferably, the period of time for light illumination is between 2 and 20 minutes.

A variety of light delivery devices may be utilized to practice the present invention. A hand manipulable light wand or fiber optic device may be used to illuminate tissue within a living body. Such fiber optic devices may include a disposable fiber optic guide provided in kit form with a solution containing a photosensitive material. Other potential light devices for use in accordance with the present invention include the devices disclosed in U.S. Pat. No. 6,159,236, entitled Expandable treatment device for photodynamic therapy and method of using same, and U.S. Pat. No. 6,048,359, entitled Spatial orientation and light sources and method of using same for medical diagnosis and photodynamic therapy, both incorporated by reference in their entireties herein.

Repeat administrations of a treatment protocol may also be necessary or desired, including repeat administrations of photosensitive functionalized fullerenes and light activation. The repeat administrations may include different photosensitive materials and/or different compounds than earlier administered. Repeat administrations of the treatment protocol may continue for a period of time.

In general, an effective amount of a functionalized fullerene compound will be in the range of from about 0.1 to about 10 mg by injection or from about 5 to about 100 mg orally. Such dosages may vary, for example, depending on whether multiple administrations are given, tissue type and route of administration, the condition of the individual, the desired objective and other factors known to those of skill in the art.

Compositions of the present invention are administered by a mode appropriate for the form of composition. Available routes of administration include subcutaneous, intramuscular, intraperitoneal, intradermal, oral, intranasal, intrapulmonary (i.e., by aerosol), intravenously, intramuscularly, subcutaneously, intracavity, intrathecally or transdermally, alone or in combination with other pharmaceutical agents. Therapeutic compositions of photosensitizers are often administered by injection or by gradual perfusion, or by topical application to the skin or mucous membrane in need of treatment.

Compositions for oral, intranasal, or topical administration can be supplied in solid, semi-solid or liquid forms, including tablets, capsules, powders, liquids, and suspensions. Compositions for injection can be supplied as liquid solutions or suspensions, as emulsions, or as solid forms suitable for dissolution or suspension in liquid prior to injection. For administration via the respiratory tract, a preferred composition is one that provides a solid, powder, or liquid aerosol when used with an appropriate aerosolizer device. Although not required, compositions are preferably supplied in unit dosage form suitable for administration of a precise amount. Also contemplated by this invention are slow-release or sustained release forms, whereby a relatively consistent level of the active compound are provided over an extended period.

Another method of administration is intravascular, for instance by direct injection into the blood vessels of the infected tissue or surrounding area.

Further, it may be desirable to administer the compositions locally to the area in need of treatment. This can be achieved, for example, by local infusion during surgery, by injection, by means of a catheter, or by means of an implant, said implant being of a porous, non-porous, or gelatinous material, including membranes, such as silastic membranes, or fibers. A suitable such membrane is Gliadel® provided by Guilford Pharmaceuticals Inc.

Combination Therapies

The fullerene PDT compositions of this invention can be administered as the sole pharmaceutical agent or in combination with one or more other pharmaceutical agents where the combination causes no unacceptable adverse effects. For example, the fullerene PDT compositions of this invention can be combined with known anti-hyper-proliferative or other indication agents, and the like, as well as with admixtures and combinations thereof.

The additional pharmaceutical agent can be aldesleukin, alendronic acid, alfaferone, alitretinoin, allopurinol, aloprim, aloxi, altretamine, aminoglutethimide, amifostine, amrubicin, amsacrine, anastrozole, anzmet, aranesp, arglabin, arsenic trioxide, aromasin, 5-azacytidine, azathioprine, BCG or tice BCG, bestatin, betamethasone acetate, betamethasone sodium phosphate, bexarotene, bleomycin sulfate, broxuridine, bortezomib, busulfan, calcitonin, campath, capecitabine, carboplatin, casodex, cefesone, celmoleukin, cerubidine, chlorambucil, cisplatin, cladribine, cladribine, clodronic acid, cyclophosphamide, cytarabine, dacarbazine, dactinomycin, DaunoXome, decadron, decadron phosphate, delestrogen, denileukin diftitox, depo-medrol, deslorelin, dexrazoxane, diethylstilbestrol, diflucan, docetaxel, doxifluridine, doxorubicin, dronabinol, DW-166HC, eligard, elitek, ellence, emend, epirubicin, epoetin alfa, epogen, eptaplatin, ergamisol, estrace, estradiol, estramustine phosphate sodium, ethinyl estradiol, ethyol, etidronic acid, etopophos, etoposide, fadrozole, farston, filgrastim, finasteride, fligrastim, floxuridine, fluconazole, fludarabine, 5-fluorodeoxyuridine monophosphate, 5-fluorouracil (5-FU), fluoxymesterone, flutamide, formestane, fosteabine, fotemustine, fulvestrant, gammagard, gemcitabine, gemtuzumab, gleevec, gliadel, goserelin, granisetron HCl, histrelin, hycamtin, hydrocortone, eyrthro-hydroxynonyladenine, hydroxyurea, ibritumomab tiuxetan, idarubicin, ifosfamide, interferon alpha, interferon-alpha 2, interferon alfa-2α, interferon alfa-2B, interferon alfa-n1, interferon alfa-n3, interferon beta, interferon gamma-1α, interleukin-2, intron A, iressa, irinotecan, kytril, lentinan sulphate, letrozole, leucovorin, leuprolide, leuprolide acetate, levamisole, levofolinic acid calcium salt, levothroid, levoxyl, lomustine, lonidamine, marinol, mechlorethamine, mecobalamin, medroxyprogesterone acetate, megestrol acetate, melphalan, menest, 6-mercaptopurine, Mesna, methotrexate, metvix, miltefosine, minocycline, mitomycin C, mitotane, mitoxantrone, Modrenal, Myocet, nedaplatin, neulasta, neumega, neupogen, nilutamide, nolvadex, NSC-631570, OCT-43, octreotide, ondansetron HCl, orapred, oxaliplatin, paclitaxel, pediapred, pegaspargase, Pegasys, pentostatin, picibanil, pilocarpine HCl, pirarubicin, plicamycin, porfimer sodium, prednimustine, prednisolone, prednisone, premarin, procarbazine, procrit, raltitrexed, rebif, rhenium-186 etidronate, rituximab, roferon-A, romurtide, salagen, sandostatin, sargramostim, semustine, sizofuran, sobuzoxane, solu-medrol, sparfosic acid, stem-cell therapy, streptozocin, strontium-89 chloride, synthroid, tamoxifen, tamsulosin, tasonermin, tastolactone, taxotere, teceleukin, temozolomide, teniposide, testosterone propionate, tested, thioguanine, thiotepa, thyrotropin, tiludronic acid, topotecan, toremifene, tositumomab, trastuzumab, treosulfan, tretinoin, trexall, trimethylmelamine, trimetrexate, triptorelin acetate, triptorelin pamoate, UFT, uridine, valrubicin, vesnarinone, vinblastine, vincristine, vindesine, vinorelbine, virulizin, zinecard, zinostatin stimalamer, zofran, ABI-007, acolbifene, actimmune, affinitak, aminopterin, arzoxifene, asoprisnil, atamestane, atrasentan, BAY 43-9006 (sorafenib), avastin, CCI-779, CDC-501, celebrex, cetuximab, crisnatol, cyproterone acetate, decitabine, DN-101, doxorubicin-MTC, dSLIM, dutasteride, edotecarin, eflomithine, exatecan, fenretinide, histamine dihydrochloride, histrelin hydrogel implant, holmium-166 DOTMP, ibandronic acid, interferon gamma, intron-PEG, ixabepilone, keyhole limpet hemocyanin, L-651582, lanreotide, lasofoxifene, libra, lonafarnib, miproxifene, minodronate, MS-209, liposomal MTP-PE, MX-6, nafarelin, nemorubicin, neovastat, nolatrexed, oblimersen, onco-TCS, osidem, paclitaxel polyglutamate, pamidronate disodium, PN-401, QS-21, quazepam, R-1549, raloxifene, ranpirnase, 13-cis-retinoic acid, satraplatin, seocalcitol, T-138067, tarceva, taxoprexin, thymosin alpha 1, tiazofurine, tipifarnib, tirapazamine, TLK-286, toremifene, TransMID-107R, valspodar, vapreotide, vatalanib, verteporfin, vinflunine, Z-100, zoledronic acid or combinations thereof.

Optional anti-hyper-proliferative agents which can be added to the composition include but are not limited to compounds listed on the cancer chemotherapy drug regimens in the 11th Edition of the Merck Index, (1996), which is hereby incorporated by reference, such as asparaginase, bleomycin, carboplatin, carmustine, chlorambucil, cisplatin, colaspase, cyclophosphamide, cytarabine, dacarbazine, dactinomycin, daunorubicin, doxorubicin (adriamycine), epirubicin, etoposide, 5-fluorouracil, hexamethylmelamine, hydroxyurea, ifosfamide, irinotecan, leucovorin, lomustine, mechlorethamine, 6-mercaptopurine, mesna, methotrexate, mitomycin C, mitoxantrone, prednisolone, prednisone, procarbazine, raloxifen, streptozocin, tamoxifen, thioguanine, topotecan, vinblastine, vincristine, and vindesine.

Other anti-hyper-proliferative agents suitable for use with the composition of the invention include but are not limited to those compounds acknowledged to be used in the treatment of neoplastic diseases in Goodman and Gilman's The Pharmacological Basis of Therapeutics (Ninth Edition), editor Molinoff et al., publ. by McGraw-Hill, pages 1225-1287, (1996), which is hereby incorporated by reference, such as aminoglutethimide, L-asparaginase, azathioprine, 5-azacytidine cladribine, busulfan, diethylstilbestrol, 2′,2′-difluorodeoxycytidine, docetaxel, erythrohydroxynonyl adenine, ethinyl estradiol, 5-fluorodeoxyuridine, 5-fluorodeoxyuridine monophosphate, fludarabine phosphate, fluoxymesterone, flutamide, hydroxyprogesterone caproate, idarubicin, interferon, medroxyprogesterone acetate, megestrol acetate, melphalan, mitotane, paclitaxel, pentostatin, N-phosphonoacetyl-L-aspartate (PALA), plicamycin, semustine, teniposide, testosterone propionate, thiotepa, trimethylmelamine, uridine, and vinorelbine.

Other anti-hyper-proliferative agents suitable for use with the composition of the invention include but are not limited to other anti-cancer agents such as epothilone and its derivatives, irinotecan, raloxifen and topotecan.

Generally, the use of cytotoxic and/or cytostatic agents in combination with a compound or composition of the present invention will serve to:

(1) yield better efficacy in reducing the growth of a tumor or even eliminate the tumor as compared to administration of either agent alone,

(2) provide for the administration of lesser amounts of the administered chemotherapeutic agents,

(3) provide for a chemotherapeutic treatment that is well tolerated in the patient with fewer deleterious pharmacological complications than observed with single agent chemotherapies and certain other combined therapies,

(4) provide for treating a broader spectrum of different cancer types in mammals, especially humans,

(5) provide for a higher response rate among treated patients,

(6) provide for a longer survival time among treated patients compared to standard chemotherapy treatments,

(7) provide a longer time for tumor progression, and/or

(8) yield efficacy and tolerability results at least as good as those of the agents used alone, compared to known instances where other cancer agent combinations produce antagonistic effects.

The invention will be more fully understood by reference to the following examples. These examples, however, are merely intended to illustrate the embodiments of the invention and are not to be construed to limit the scope of the invention.

### EXAMPLES

### Example 1

### Synthesis of Nonionic Fullerene Derivatives

This Example describes the synthesis of a series of functionalized nonionic C₆₀ fullerene derivatives with one, two, or three polar diserinol groups (e.g., NI1, NI2, NI3, as shown in Table 2, supra).

This synthesis was carried out as described below and shown in Scheme 1.

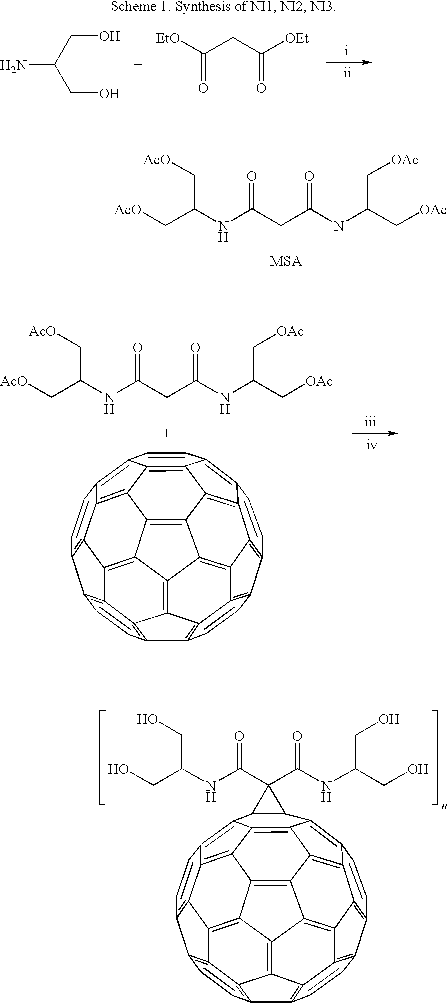

Serinol (2.05 equivalents) and diethylmalonate (1 equivalent) were reacted at 200° C. for 45 minutes in an open tube. Then acetic anhydride (4.1 equivalents) and pyridine (4.1 equivalents) were added and stirred for 18 hours at room temperature. The product termed MSA thus obtained was recrystallized using a mixture of hexane and ethyl acetate.

Purified C₆₀ (200 mg, 0.28 mmol) was dissolved in toluene (250 ml) by sonicating for 10 minutes and nitrogen was purged for 30 minutes. Then CB₄ (46.1 mg, 0.14 mmol) as a solid directly, MSA (58.2 mg. 0.14 mmol) in acetone (3 ml), and 1,8-Diazabicyclo [5.4.0]undec-7-ene (31.7 mg, 0.21 mmol) in toluene (5 ml) were added. The reaction mixture as stirred at room temperature for 4.5 hours under nitrogen atmosphere. Solvents were removed on a rotavap under vacuum. The product was dissolved in a minimum amount of chloroform and loaded onto a silica gel column (1 in×9 in) and eluted with dichloromethane containing 0-2% methanol to collect pure N11, N12 and N13. The compounds were characterized by matrix assisted laser desorption ionization mass spectrometry (MALDI-MS) as follows: N11—calculated mass 1137.02 and observed mass 1137.56; N12—calculated mass 1553.40 and observed mass 1153.77; N13—calculated mass 1969.78 and observed mass 1970.26.

NMR data were obtained for C₆₀(MSA)-protected NI1: ¹H NMR (400 MHz, CDCl₃, TMS ref.) δ (ppm) 2.10 (s, 12H, CH₃), 4.34-4.41 (m, 8H, CH₂), 4.68-4.72 (m, 2H, CH), 7.37 (br d, J 56.4 Hz, 2H, NH).

Deprotection of —OH groups was achieved by treating NI1-3 with an excess of potassium carbonate in methanol and deionized water at room temperature for 90 minutes. Potassium ions were removed by adding strong cation exchange resin (Biorad AG MP-50W, treated with 1M HCl) to the reaction mixture until the pH reached 7. The solution was filtered and solvents were removed on a rotavap to obtain pure NI1, NI2, and NI3.

### Example 2

### Synthesis of Cationic Fullerene Derivatives

This Example describes a scheme for synthesis of cationic fullerene derivatives (e.g., CI1, CI2, and CI3, as illustrated in Table 2, supra).

The synthesis of compounds CI1-3 was carried out using published procedures (Wharton, T., Kini, V. U., Mortis, R. A., and Wilson, L. J. (2001). New non-ionic, highly water-soluble derivatives of C60 designed for biological compatibility. Tetrahedron Lett. 42, 5159-5162, Wharton, T., and Wilson, L. J. (2002). Highly-iodinated fullerene as a contrast agent for X-ray imaging. Bioorg. Med. Chem. 10, 3545-3554, Maggini, M., Scorrano, G., and Prato, M. (1993). Addition of azomethine ylides to C60: synthesis, characterization, and functionalization of fullerene pyrrolidines. J. Am. Chem. Soc. 115, 9798-9799 and Cassell, A. M., Scrivens, W. A., and Tour, J. M. (1998). Assembly of DNA/fullerene hybrid materials. Angew. Chem. Int. Ed. Engl. 37, 1528-1530.) with modifications as described below, and illustrated in Scheme 2.

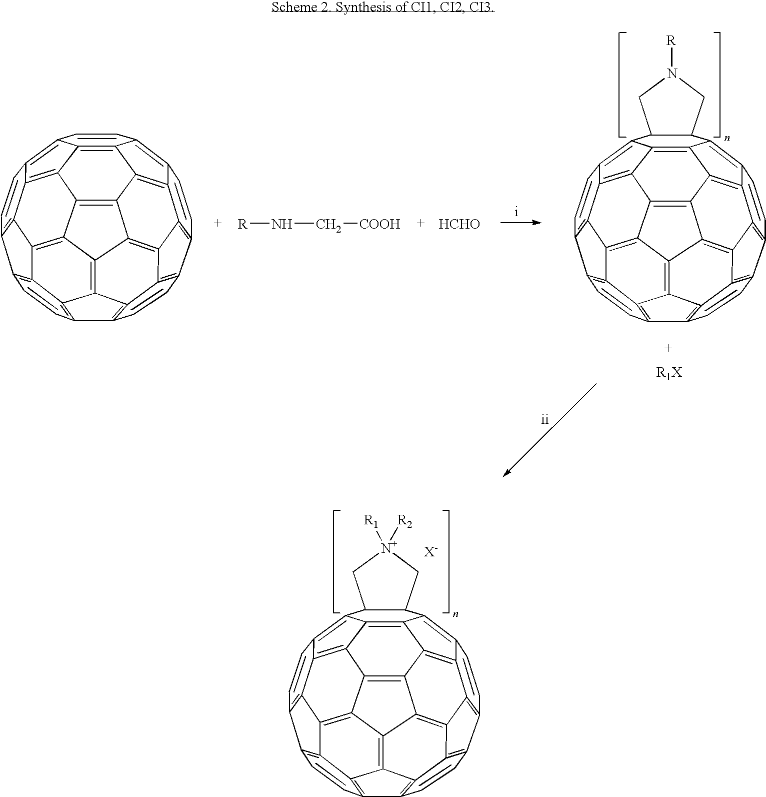

Purified C₆₀ (200 mg, 0.28 mmol) was dissolved in toluene (260 ml) by sonicating for 5 minutes. To this solution were added sarcosine (50.8 mg, 0.57 mmol) and paraformaldehyde (40.9 mg, 1.36 mmol) for CI1; sarcosine (63.5 mg, 0.71 mmol) and paraformaldehyde (35.79 mg, 1.19 mmol) for CI2; or sarcosine (88.9 mg, 1.0 mmol) and paraformaldehyde (46.0 mg, 1.53 mmol) for CI3, as solids directly. The reaction mixture was refluxed for 2 hours for CI1; overnight for CI2; and 3 hours for CI3. Solvents were removed on a rotavap under vacuum.

The product was dissolved in a minimum amount of toluene and loaded onto a silica gel column (1 in ×9 in) packed with toluene and eluted with toluene containing 0-5% acetone to collect pure CI1, CI2, or CI3, with yields of 30-40% purity. The purity of the compounds in terms of nono-, bis-, and tris-substitutions was confirmed by thin layer chromatography (TLC).

Methylation of CI1, CI2, or CI3 was carried out by dissolving the compounds in a large excess of methyl iodide (1 ml per 20 mg CI1-3) and stirring for 48-72 hours at room temperature (or 7 days in the case of CI3). Pure methylated products CI1, CI2, or CI3 were precipitated by adding hexanes, and the precipitates were collected, washed with toluene and dichloromethane, and dried. The compounds were characterized by electrospray mass spectrometry (ES-MS) as follows: CI1—calculated mass 792.08 and observed mass 792.04; CI2—calculated mass 864.16 and observed mass 432.05 (M²⁺); and CI3—calculated mass 936.24 and observed mass 312.08 (M³⁺).

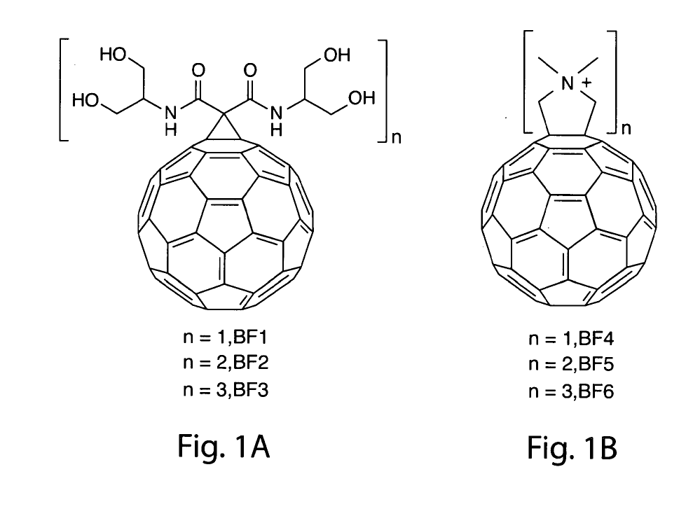

NMR data were obtained for CI1 as follows: ¹H NMR (400 MHz, 2:3 CDCl₃:DMSO-d₆, TMS ref.) δ (ppm) 4.08 (s, 6H, CH₃), 5.72 (s, 4H, CH₂). Referring to FIG. 1, UV-visible absorption spectra of the compounds were recorded in 1:9 DMSO:water at a concentration of 10 mM. More particularly, FIG. 1 shows UV-Visible absorption spectra of CI1-3 and toluidine blue 0 (TBO) at 10 μM concentration in 1:9 DMSO:water.

### Example 3

### Synthesis of Nitrogenous Fullerene Derivatives

This Example describes a scheme for synthesis of nitrogenous fullerene derivatives (e.g., N1 as illustrated in Table 2, supra).

The synthesis of compound N1 was carried out as described below and illustrated in Scheme 3.

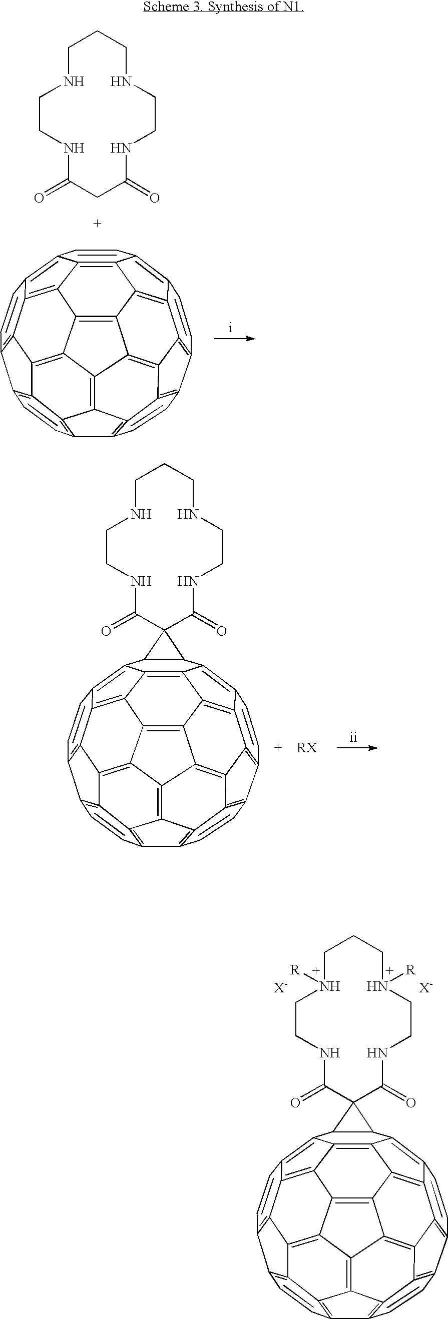

Purified C₆₀ (360 mg, 0.5 mmol) was dissolved in toluene (180 ml) by sonicating for 30 minutes and nitrogen was purged for 15 minutes. Then CBr₄ (83 mg, 0.25 mmol) as solid directly, 1,4,8,11-tetraazacyclotetradecane-5,7-dione (57 mg, 0.25 mmol) in methanol (1 ml) and toluene (9 ml), and DBU (57 mg, 0.375 mmol) in toluene (10 ml) were added. The reaction mixture was stirred at room temperature for 24 hours under nitrogen atmosphere. The product N1 was precipitated and filtered, washed with toluene and dried.

Methylation of N1 was carried out by suspending in a large excess of methyl iodide and stirring for 72 hours at room temperature. The methylated product N1 was precipitated and which was collected and washed with toluene and dichloromethane, and dried.

### Example 4

### Synthesis of Cationic CI4 and CI5 Fullerene Derivatives

The synthesis of cationic compounds CI4 and CI5 was carried out as described below, and illustrated in Scheme 4.

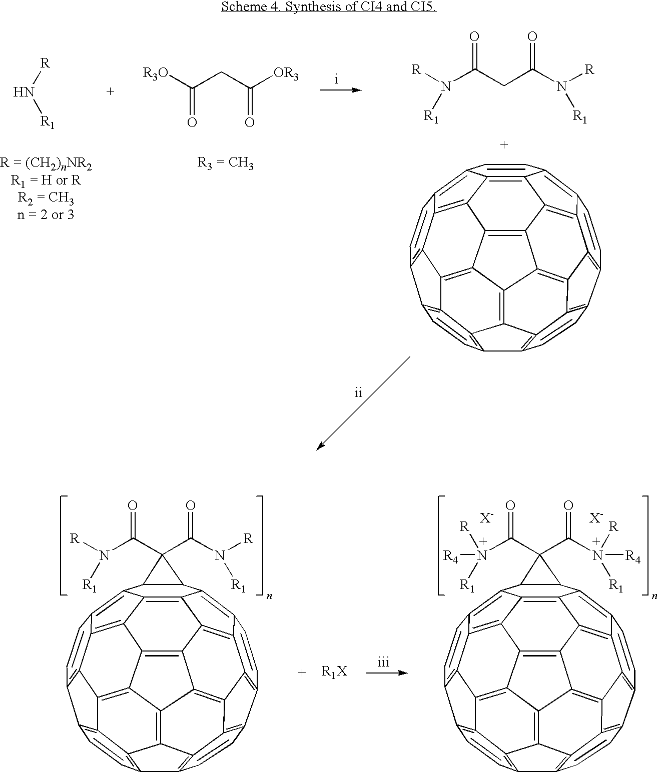

For synthesis of diquat-21, (CH₃)₂N(CH₂)₂NH₂ (2.05 equivalents) and dimethylmalonate (1 equivalent) were dissolved in toluene and reacted at 100° C. for 2 hours. The solvents were removed on a rotavap and added hexanes. The product was cooled in a refrigerator overnight and filtered. The product obtained as a pink waxy solid.

For synthesis of diquat-31, (CH₃)₂N(CH₂)₂NH₂ (2.05 equivalents) and dimethylmalonate (1 equivalent) were reacted at 120° C. for 2 hours. The solvents were removed on a rotavap. The product was obtained as a high viscous pale yellow liquid after vacuum drying for 60 hours at 20° C.

For synthesis of CI4 and CI5, purified C₆₀ (360 mg, 0.5 mmol) was dissolved in toluene (180 ml) by sonicating for 15 minutes and nitrogen was purged for 15 minutes. Then CBr₄ (83 mg, 0.25 mmol) as a solid directly, diquat (0.25 mmol) in toluene (5 ml), toluene (9 ml), and DBU (57 mg, 0.375 mmol) in toluene (10 ml) were added. The reaction mixture was stirred at room temperature for 4 hours under nitrogen atmosphere. The product C₆₀-diquat was precipitated and filtered, washed with toluene, and dried.

Methylation of C₆₀-diquat was carried out by dissolving the compounds in a large excess of methyl iodide and stirring for 72 hours at room temperature. The methylated product was precipitated and collected, washed with toluene and dichloromethane, and dried. The compounds were characterized by electrospray mass spectrometry (ES-MS) as follows. CI4-calculated mass 993.03 and observed mass 496.09 (M²⁺); CI5— calculated mass 1021.08 and observed mass 510.11 (M²⁺).

### Example 5

### Absorption Spectra of Derivatized Fullerenes

This Example describes one aspect of the characterization (determination of absorption spectra) of functionalized fullerenes NI1-3 and CI1-3 of the invention.

Functionalized fullerenes NI1-3 and CI1-3 were prepared as described above. There are eight possible regioisomers of the bis-substituted fullerenes and 46 possible regioisomers of the tris-substituted fullerenes. It was not practical to separate these mixtures of regioisomers into individual pure compounds; therefore, NI2 and NI3, and CI2 and CI3 were studied as mixtures of regioisomers. The identity of the compounds, however, was confirmed by mass spectrometry, giving molecular ions identical to those calculated. The proton and CI3 NMR spectra of the immediate precursors of BF1 and BF4 have been reported (Wharton, T., Kini, V. U., Mortis, R. A., and Wilson, L. J. (2001). New non-ionic, highly water-soluble derivatives of C60 designed for biological compatibility. Tetrahedron Lett. 42, 5159-5162, Maggini, M., Scorrano, G., and Prato, M. (1993). Addition of azomethine ylides to C60: synthesis, characterization, and functionalization of fullerene pyrrolidines. J. Am. Chem. Soc. 115, 9798-9799).

The absorption spectra of CI1-3 and TBO, all at the same concentration of 10 μM in DMSO:water (i.e., 1:9), are shown in FIG. 1. The overall extinction coefficients of the fullerenes were in the following order: CI1>CI2>CI3. The shoulder in the UVA range moved from 340 nm for CI1 to 310 nm for CI2 and disappeared altogether for CI3 (FIG. 1).

### Example 6

### Distribution Coefficients of Derivatized Fullerenes

This Example describes studies performed to determine the distribution coefficients of fullerenes NI1-3 and CI1-3 of the invention.

Each compound was dissolved in a minimum amount of DMSO: CI1 (0.9 mg in 200 μl), CI2 (5.3 mg in 200 μl), CI3 (5.4 mg in 200 μl). Ten ml of DI water and 10 ml of 1-octanol were added in each compound and vigorously shaken for 2 min. and the vials of the compounds were settled down overnight. The phases were separated and UV-spectra of each phase were analyzed. Distribution coefficient of each compound was determined using absorbance of aqueous phases and organic phases at 330 nm.

The results of these determinations are presented in Table 2. Referring to Table 2, it will be appreciated that the hydrophilic character of fullerene derivatives increases with increasing number of cationic functional groups, whereas hydrophilicity decreases with increasing number of serinol groups.

TABLE 2
Octanol-water partition constants (K_{ow})
of Fullerene Derivatives NI1-3 and CI1-3.

| Compound | NI1 | NI2 | NI3 | CI1 | CI2 | CI3 |
|---|---|---|---|---|---|---|
| K_{ow} | 0.025 | 0.032 | 0.078 | 140.80 | 1.28 | 0.37 |
| LogK_{ow} | −1.61 | −1.49 | −1.11 | 2.15 | 0.11 | −0.43 |

### Example 7

### Determining Phototoxicity, Apoptosis Activity, Intracellular Reactive Oxygen Species and Photophysical Properties of Derivatized Fullerenes

This Example describes exemplary materials and methods useful for testing derivatized fullerenes prepared in accordance with the invention.

1. Cell Lines and Culture Conditions.

A panel of murine cancer cells lines: J774 reticulum sarcoma [30], Lewis lung carcinoma (LLC) [31], and colon adenocarcinoma (CT26) [32] was obtained from ATCC (Manassas, Va.). The cells were cultured in RPMI medium with L-glutamine and NaHCO₃ supplemented with 10% heat inactivated fetal bovine serum, penicillin (100 U/mL) and streptomycin (100 μg/mL) (Sigma, St Louis, Mo.) at 37° C. in 5% CO2 humidified atmosphere in 75 cm2 flasks (Falcon, Invitrogen, Carlsbad, Calif.).

2. Photosensitizers and Light Sources

For illumination of cells a white light source (Lumacare, Newport Beach, Calif.) fitted with a light guide containing a band pass filter (400-700 nm) was used and adjusted to give a uniform spot of 4 cm in diameter with an irradiance of 150 mW/cm2 as measured with a power meter (model DMM 199 with 201 Standard head, Coherent, Santa Clara, Calif.).

3. Photodynamic Inactivation Studies

When the cells reached 80% confluence, they were washed with PBS and harvested with 2 mL of 0.25% trypsin-EDTA solution (Sigma). Cells were then centrifuged and counted in trypan blue to ensure viability and plated at density of 5000/well in flat-bottom 96 well plates (Fisher Sci, Pittsburgh, Pa.). Cells were allowed 24 h to attach.

On the following day dilutions of the fullerenes were prepared in complete RPMI medium and added to the cells at 204 concentration for 24 h incubation as described. Prior to illumination the fullerene solution was removed and fresh complete medium was replaced and the illumination (150 mW/cm2 white light, 1-80 J/cm2) was performed. The light spot covered 9 wells which were considered as one experimental group. All wells in a group were illuminated at the same time. The absolute control, DMSO control and light control groups received; nothing, DMSO (0.0032%) and light (maximal fluence) respectively.

Following PDT treatment the cells were returned to the incubator overnight and the phototoxicity was measured using a 4 h MTT assay read at 560 nm using a microplate spectrophotometer (Spectra Max 340 PC, Molecular Devices, Sunnyvale, Calif.). Each experiment was repeated 2-4 times.

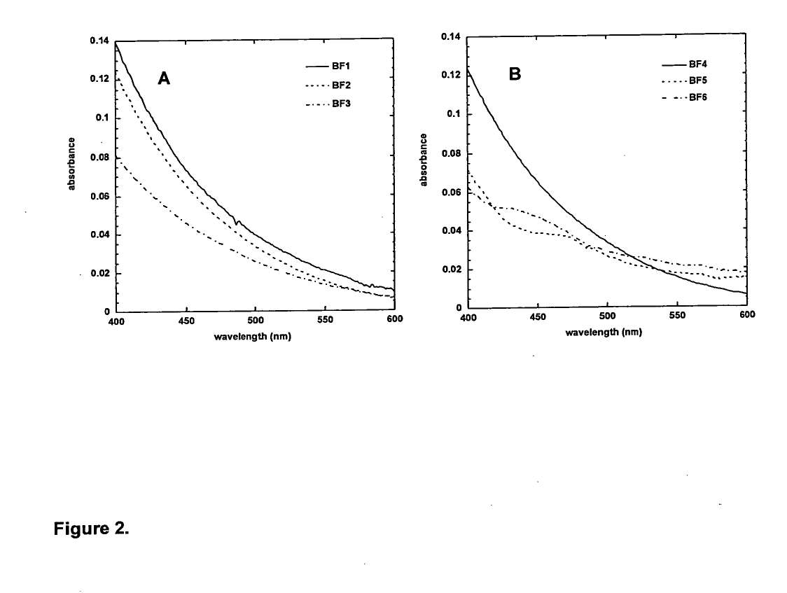

FIG. 2A shows the fluence-dependent loss of mitochondrial activity for the 6 fullerenes on LLC lung cancer cells. FIG. 2B shows the PDT killing of the J774 reticulum sarcoma cell line that has the characteristics of macrophages in tissue culture. FIG. 2C shows the results of the PDT killing of the third mouse cancer cell line tested, colon adenocarcinoma CT26.

4. Apoptosis Induction Assay

The induction of apoptosis by fullerene-mediated PDT was measured by a fluorescence assay using Ac-DEVD-AFC, a caspase fluorescent substrate [33]. The results were normalized to the content of protein in the sample.

Briefly cells were treated with PDT sufficient to kill 80% of cells (5 J/cm2 for J774, 20 J/cm2 for LLC and 80 J/cm2 for CT26). Following PDT samples were collected at 1, 2, 4, 6, 12, and 24 h and centrifuged. The pellet was resuspended in 100 μL of lysis buffer [34] containing protease inhibitor and subjected to 3-4 cycles of freezing and thawing. Then 50 μL, of each sample was transferred to separate wells and 50 μL of 2× reaction buffer was added together with Ac-DEVD-AFC (final concentration 50 μM). Samples were incubated in the dark for 1 h at 37° C. and fluorescence was measured (excitation 400 nm, emission 505 nm). The protein per sample was measured with bicinchoninic acid protein assay [35].

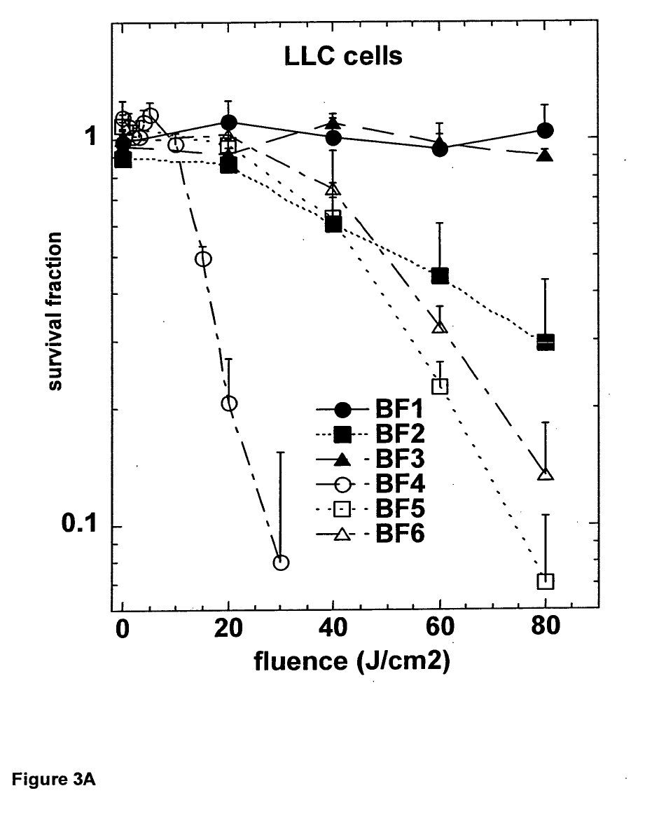

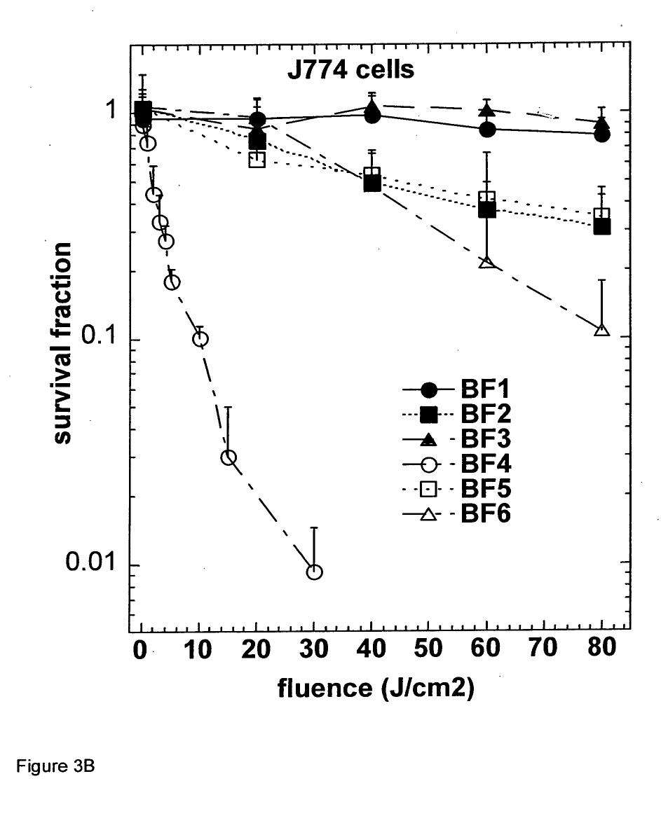

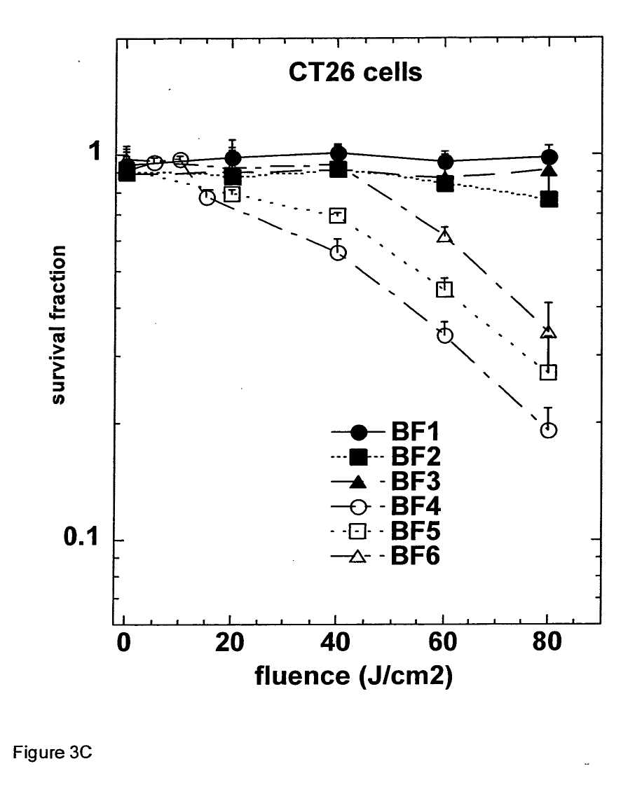

FIG. 3 shows the time course of apoptosis in CT26 cells after incubation with 2 μM BF4 or BF6 and illumination with 80 J/cm2 of light.

5. Intracellular Reactive Oxygen Species

J774 cells were incubated with 5 μM BF4 for 24 h and on the next day 5 μg/ml of 5- (and -6)-chloromethyl-2′-7′-dichlorodihydrofluoresceine diacetate, (CMH2DCFDA, Molecular Probes, Invitrogen) in complete medium was added and incubated for 30 min at 37° C., then cells were washed with PBS and 5 J/cm2 of 405 nm laser light (Nichia Corp, Detroit, Mich.) was delivered. Five to 10 min later a Leica DMR confocal laser fluorescence microscope (Leica Mikroskopie and Systeme GmbH, Wetzler, Germany) with excitation by a 488 nm argon laser and emission with a 530 nm+/−10 nm bandpass filter, and a 63×1.20 water immersion lens was used to image the cells at a resolution of 1024×1024 pixels. Images were acquired using TCS NT software (Version 1.6.551, Leica Lasertechnik, Heidelberg, Germany).

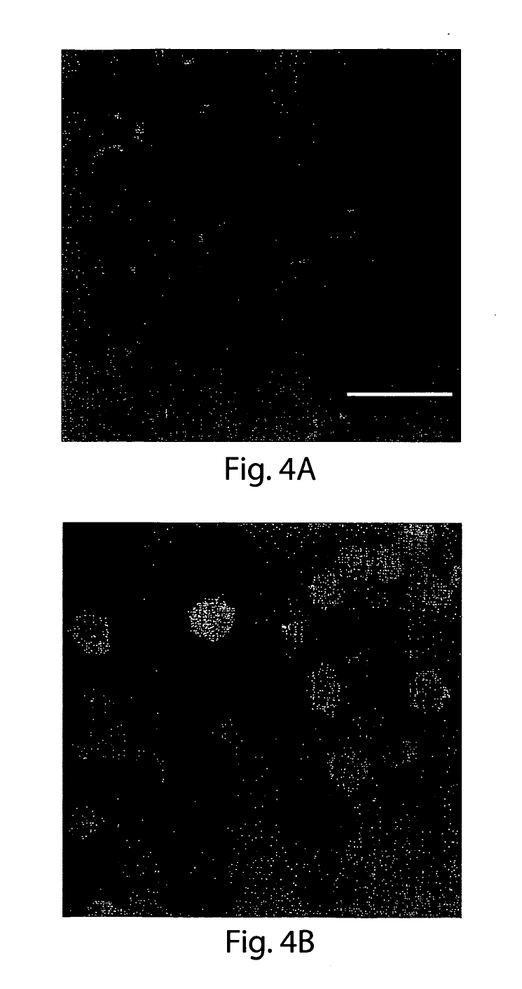

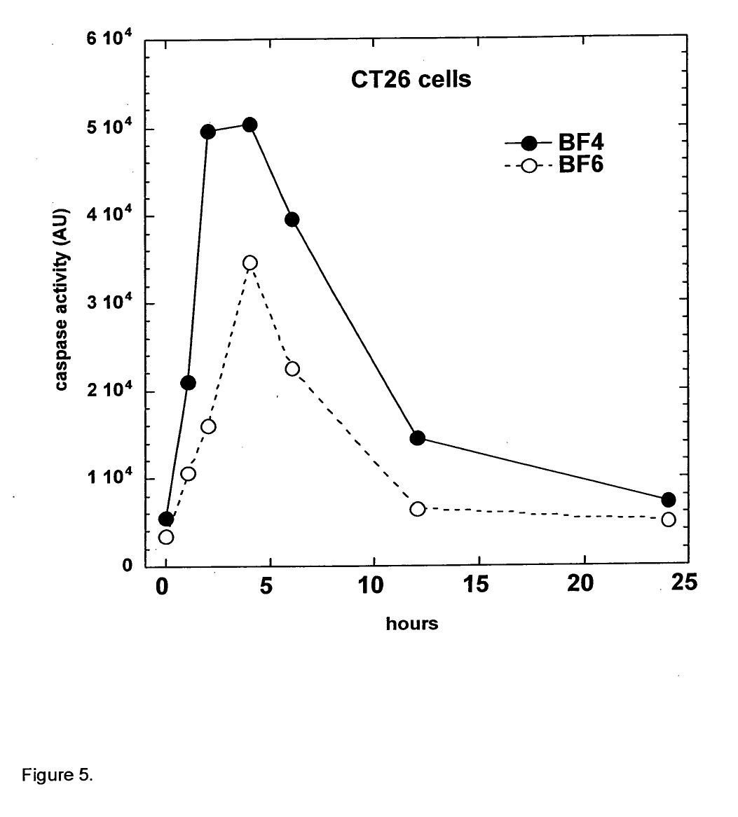

FIG. 4 shows the fluorescence micrographs of illuminated J774 cells that had been incubated with either the H2DCFDA probe without fullerene (panel A) or BF4 for 24 hours followed by the probe (panel B). There is only trace green fluorescence visible in cells with probe alone, while the cells that had both fullerene and probe demonstrated a large increase in fluorescence that was evenly distributed throughout the cells, consistent with a diffusible species such as H₂O₂ having been produced during illumination.

6. Photophysical Studies

Riboflavin, dimethyl sulfoxide (DMSO), histidine, sodium azide, 5,5-dimethyl-1-pyrroline-N-oxide (DMPO), nicotinamide adenine dinucleotide (NADH), deuterated methanol (CH3OD) and deuterated water (D2O) were from Sigma-Aldrich. All chemicals were used as supplied except for DMPO, which was purified by vacuum distillation. The spin probe, 4-protio-3-carbamoyl-2,2,5,5,-tetraperdeuteromethyl-3-pyrroline-1-yloxy (mHCTPO) was a gift from Professor H. J. Halpern (University of Chicago, Ill.).

Photo-dependent oxygen uptake kinetics in irradiated samples were measured by ESR oximetry [36, 37]. A sample in a mixture of water and DMSO (1:3; v/v) containing fullerenes, and 0.1 mM mHCTPO as the nitroxide spin probe was placed in a flat quartz cell (0.25 mm) in a resonant cavity and illuminated with white light (390-700 nm) from a 300 W high-pressure xenon lamp (Perkin-Elmer, Fremont, Calif.) equipped with a combination of filters.

To the samples, 1 mM NADH or 2 mM histidine with and without 5 mM sodium azide were added to determine their effects on photoconsumption of oxygen. Instrument settings were: microwave power—1 mW; modulation amplitude—0.1 G; sweep—4 G; time constant—20.48 ms

Formation of O₂*⁻ was detected as described previously [36]. The DMPO spin probe (0.1M) was used as a spin trap for the detection of superoxide anion [38, 39].

The sample in a 0.25 mm quartz cell was illuminated within the resonant cavity as described above. Singlet oxygen phosphorescence at 1270 nm was monitored by a nitrogen-cooled germanium detector (Model EO-817, North Coast Scientific Corp, Santa Rosa, Calif.). Photoexcitation of the sample studied was induced by a 5 ns 355 nm laser pulse from a Q-switched Nd:YAG laser (Continuum Surelite II, Santa Clara, Calif.) equipped with an optical parametric oscillator (Opotek, Carlsbad, Calif.). Sample in deuterated PBS (pD ˜6.9) or in deuterated methanol (CH3OD) was excited with 449 nm wavelength.

Quantum yields of singlet oxygen generation were determined using riboflavin as a standard (Φrbfl=0.51 in methanol; [40]; Φrbfl=0.49 in PBS; [41].

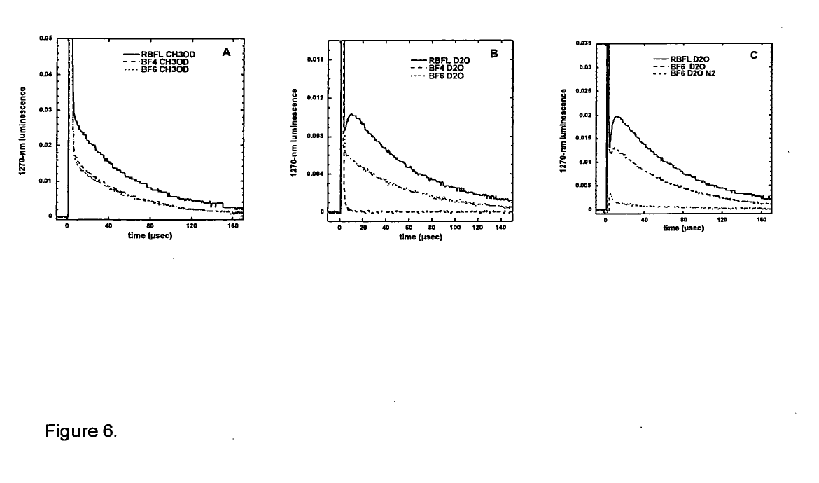

FIG. 4A shows that in an organic solvent (CH₃OD) both BF4 and BF6 gave very similar luminescence decay curves, while the curve obtained from riboflavin was somewhat larger. When the solvent was changed to an aqueous buffer, the singlet oxygen decay curve of BF4 almost disappeared, while the curves of BF6 and riboflavin remained almost unchanged (FIG. 6B). To confirm that the observed decay curves were oxygen dependent and therefore reflected the formation and decay of singlet oxygen we repeated the experiment with BF6 in aqueous buffer saturated with nitrogen and the luminescence disappeared as shown in FIG. 6C. Table 2 shows the calculated singlet oxygen quantum yields (with reference to riboflavin) from BF4 and BF6 in air saturated D₂O or CH³OD, and in O² saturated D₂O where the value for BF6 was about 50% higher than that found in air.

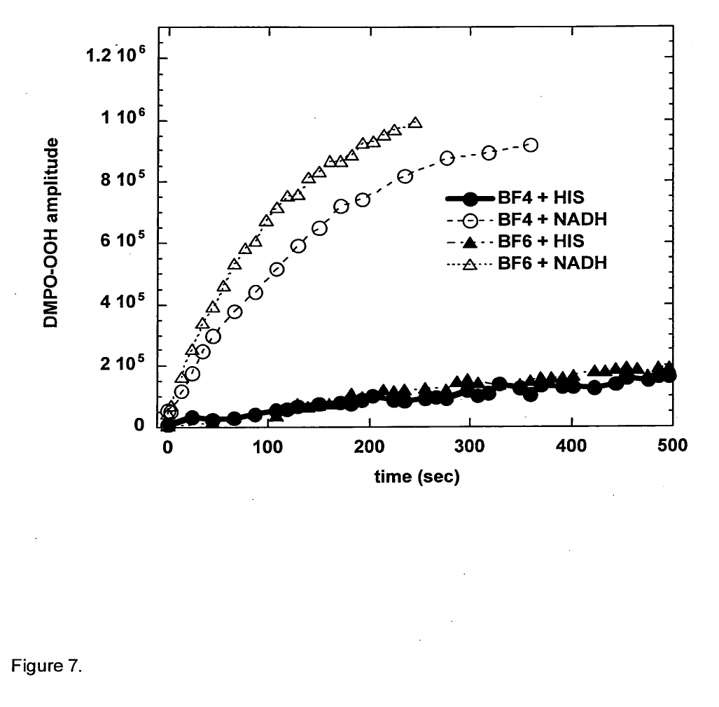

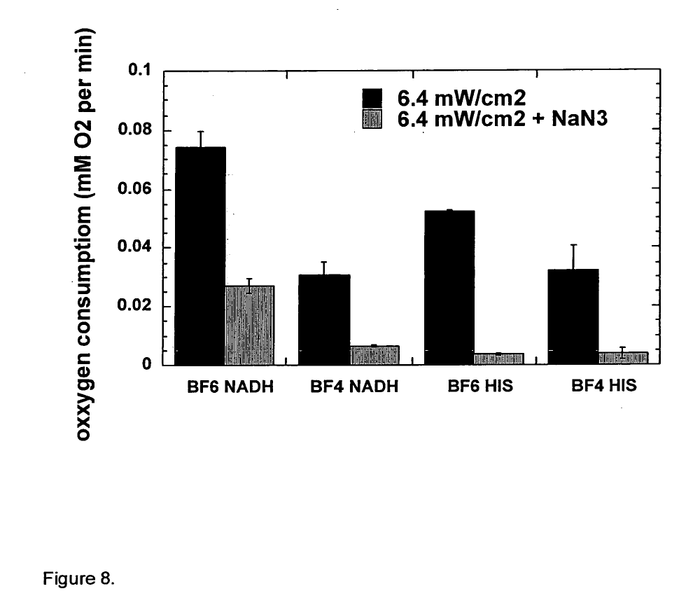

FIG. 7 shows that both BF4 and BF6 produced substantial amounts of superoxide in the presence of NADH, with BF6 giving more superoxide than BF4. The production of superoxide was sharply lower (at least ten times) in the presence of histidine

The total oxygen consumption by these two fullerenes when illuminated in 75% DMSO was measured in the presence of NADH or of histidine and the quenching effect of added sodium azide was also studied (FIG. 6).

Those skilled in the art will recognize, or be able to ascertain using no more than routine experimentation, many equivalents to the specific embodiments of the invention described herein. Such equivalents are intended to be encompassed by the following claims. All patents, published patent applications and references disclosed herein are incorporated by reference in their entireties.

### REFERENCES

It is believed that a review of the following references will appreciate understanding of the present invention. Some of these documents are referred to throughout the present disclosure by a number, as indicated below.
[1] Kroto, H. W.; Heath, J. R.; O'Brien, S. C.; Curl, R. F.; Smalley, R. E. C60: Buckminsterfullerene. Nature. 318:162-163; 1985.
[2] Jensen, A. W.; Wilson, S. R.; Schuster, D. I. Biological applications of fullerenes. Bioorg. Med Chem. 4:767-779; 1996.
[3] Bosi, S.; Da Ros, T.; Spalluto, G.; Prato, M. Fullerene derivatives: an attractive tool for biological applications. Eur J Med. Chem. 38:913-923; 2003.
[4] Dugan, L. L.; Lovett, E. G.; Quick, K. L.; Lotharius, J.; Lin, T. T.; O'Malley, K. L. Fullerene-based antioxidants and neurodegenerative disorders. Parkinsonism Relat Disord. 7:243-246; 2001.
[5] Pukhova, I.; Churilov, G. N.; Isakova, V. G.; Korets, A. I.; Titarenko, I. Study of the biological activity of the water-soluble fullerene complexes. Doklady Akademii Nauk 355:269-272; 1997.
[6] Yamago, S.; Tokuyama, H.; Nakamura, E.; Kikuchi, K.; Kananishi, S.; Sueki, K.; Nakahara, H.; Enomoto, S.; Ambe, F. In vivo biological behavior of a watermiscible fullerene: 14C labeling, absorption, distribution, excretion and acute toxicity. Chem Biol. 2:385-389; 1995.
[7] Irie, K.; Nakamura, Y.; Ohigashi, H.; Tokuyama, H.; Yamago, S.; Nakamura, E. Photocytotoxicity of water-soluble fullerene derivatives. Biosci Biotechnol Biochem. 60:1359-1361; 1996.
[8] Dolmans, D. E.; Fukumura, D.; Jain, R. K. Photodynamic therapy for cancer. Nat Rev. Cancer. 3:380-387; 2003.
[9] Brown, S. B.; Mellish, K. J. Verteporfin: a milestone in ophthalmology and photodynamic therapy. Expert Opin Pharmacother. 2:351-361; 2001.
[10] Babilas, P.; Karrer, S.; Sidoroff, A.; Landthaler, M.; Szeimies, R. M. Photodynamic therapy in dermatology—an update. Photodermatol Photoimmunol Photomed. 21:142-149; 2005.
[11] Hamano, T.; Okuda, K.; Mashino, T.; Hirobe, M.; Arakane, K.; Ryu, A.; Nashiko, S.; Nagano, T. Singlet oxygen production from fullerene derivatives: effect of sequential functionalization of the fullerene core. Chem Commun. 21-22; 1997.
[12] Yamakoshi, Y.; Sueyoshi, S.; Miyata, N. Biological activity of photoexcited fullerene. Bull Nat Inst Health Sci Japan 117:50-60; 1999.
[13] Yamakoshi, Y.; Umezawa, N.; Ryu, A.; Arakane, K.; Miyata, N.; Goda, Y.; Masumizu, T.; Nagano, T. Active oxygen species generated from photoexcited fullerene (C60) as potential medicines: O₂-* versus 1O2. J Am Chem Soc. 125:12803-12809; 2003.
[14] Ochsner, M. Photophysical and photobiological processes in the photodynamic therapy of tumours. J Photochem Photobiol B. 39:1-18; 1997.
[15] An, Y. Z.; Chen, C. B.; Anderson, J. L.; Sigman, D. S.; Foote, C. S.; Rubin, Y. Sequence-specific modification of guanosine in DNA by a C60-linked deoxyoligonucleotide: evidence for a non-singlet oxygen mechanism. Tetrahedron. 52:5179-5189; 1996.
[16] Boutorine, A. S.; Tokuyama, H.; M., T.; H., I.; E., N.; Helene, C. Fullereneoligonucleotide conjugates: photo-induced sequence-specific DNA cleavage. Angew Chem Int Ed Engl. 33:2462-2465; 1994.
[17] Liu, Y.; Zhao, Y. L.; Chen, Y.; Liang, P.; Li, L. A water-soluble beta cyclodextrin derivative possessing a fullerene tether as an efficient photodriven DNAcleavage reagent. Tetrahedron Lett. 46:2507-2511; 2005.
[18] Tokuyama, H.; Yamago, S.; Nakamura, E. Photoinduced biochemical activity of fullerene carboxylic acid. J Am Chem Soc. 115:7918-7919; 1993.
[19] Kasermann, F.; Kempf, C. Photodynamic inactivation of enveloped viruses by buckminsterfullerene. Antiviral Res. 34:65-70; 1997.
[20] Hirayama, J.; Abe, H.; Kamo, N.; Shinbo, T.; Ohnishi-Yamada, Y.; Kurosawa, S.; Ikebuchi, K.; Sekiguchi, S. Photoinactivation of vesicular stomatitis virus with fullerene conjugated with methoxy polyethylene glycol amine. Biol Pharm Bull. 22:1106-1109; 1999.
[21] Lin, Y. L.; Lei, H. Y.; Wen, Y. Y.; Luh, T. Y.; Chou, C. K.; Liu, H. S. Lightindependent inactivation of dengue-2 virus by carboxyfullerene C3 isomer. Virology. 275:258-262; 2000.
[22] Kamat, J. P.; Devasagayam, T. P.; Priyadarsini, K. I.; Mohan, H. Reactive oxygen species mediated membrane damage induced by fullerene derivatives and its possible biological implications. Toxicology. 155:55-61; 2000.
[23] Sera, N.; Tokiwa, H.; Miyata, N. Mutagenicity of the fullerene C60-generated singlet oxygen dependent formation of lipid peroxides. Carcinogenesis. 17:2163-2169; 1996.
[24] Burlaka, A. P.; Sidorik, Y. P.; Prylutska, S. V.; Matyshevska, O. P.; Golub, O. A.; Prylutskyy, Y. I.; Scharff, P. Catalytic system of the reactive oxygen species on the C60 fullerene basis. Exp. Oncol. 26:326-327; 2004.
[25] Rancan, F.; Rosan, S.; Boehm, F.; Cantrell, A.; Brellreich, M.; Schoenberger, H.; Hirsch, A.; Moussa, F. Cytotoxicity and photocytotoxicity of a dendritic C(60) mono-adduct and a malonic acid C(60) tris-adduct on Jurkat cells. J Photochem Photobiol B. 67:157-162; 2002.
[26] Yang, X. L.; Fan, C. H.; Zhu, H. S. Photo-induced cytotoxicity of malonic acid [C(60)]fullerene derivatives and its mechanism. Toxicol In Vitro. 16:41-46; 2002.
[27] Liu, J.; Ohta, S.; Sonoda, A.; Yamada, M.; Yamamoto, M.; Nitta, N.; Murata, K.; Tabata, Y. Preparation of PEG-conjugated fullerene containing Gd(3+) ions for photodynamic therapy. J Control Release. 117:104-110; 2007.
[28] Tabata, Y.; Murakami, Y.; Ikada, Y. Photodynamic effect of polyethylene glycolmodified fullerene on tumor. Jpn. J. Cancer Res. 88:1108-1116; 1997.
[29] Tegos, G. P.; Demidova, T. N.; Arcila-Lopez, D.; Lee, H.; Wharton, T.; Gali, H.; Hamblin, M. R. Cationic fullerenes are effective and selective antimicrobial photosensitizers. Chem. Biol. 12:1127-1135; 2005.
[30] Faanes, R. B.; Merluzzi, V. J.; Williams, N.; Tarnowski, G. S.; Ralph, P. Matching of chemotherapy to mouse strain and lymphoid tumor type to prevent tumorinduced suppression of specific T- and B-cell functions. Cancer Res. 39:4564-4574; 1979.
[31] Lewis, M. R.; Cole, W. H. Experimental increase of lung metastases after operative trauma (amputation of limb with tumor). AMA Arch Surg. 77:621-626; 1958.
[32] Brattain, M. G.; Strobel-Stevens, J.; Fine, D.; Webb, M.; Sarrif, A. M. Establishment of mouse colonic carcinoma cell lines with different metastatic properties. Cancer Res. 40:2142-2146; 1980.
[33] Gronda, M.; Brandwein, J.; Minden, M. D.; Pond, G. R.; Schuh, A. C.; Wells, R. A.; Messner, H.; Chun, K.; Schimmer, A. D. Assessment of the downstream portion of the mitochondrial pathway of caspase activation in patients with acute myeloid leukemia. Apoptosis. 10:1285-1294; 2005.
[34] Sane, A. T.; Bertrand, R. Distinct steps in DNA fragmentation pathway during camptothecin-induced apoptosis involved caspase-, benzyloxycarbonyl- and Ntosyl-L-phenylalanylchloromethyl ketone-sensitive activities. Cancer Res. 58:3066-3072; 1998.
[35] Sapan, C. V.; Lundblad, R. L.; Price, N. C. Colorimetric protein assay techniques. Biotechnol Appl Biochem. 29 (Pt 2):99-108; 1999.
[36] Rozanowska, M.; Jarvis-Evans, J.; Korytowski, W.; Boulton, M. E.; Burke, J. M.; Sarna, T. Blue light-induced reactivity of retinal age pigment. In vitro generation of oxygen-reactive species. J Biol. Chem. 270:18825-18830; 1995.
[37] Halpern, H. J.; Peric, M.; Nguyen, T. D.; Spencer, D. P.; Teicher, B. A.; Lin, Y. J.; Bowman, M. K. Selective isotopic labeling of a nitroxide spin label to enhance sensitivity for T2 oxymetry. J Magn Reson. 90:40-51; 1990.
[38] Finkelstein, E.; Rosen, G. M.; Rauckman, E. J. Spin trapping of superoxide and hydroxyl radical: practical aspects. Arch Biochem Biophys. 200:1-16; 1980.
[39] Finkelstein, E.; Rosen, G. M.; Rauckman, E. J.; Paxton, J. Spin trapping of superoxide. Mol Pharmacol. 16:676-685; 1979.
[40] Sikorska, E.; Khmelinskii, I.; Komasa, A.; Koput, J.; Ferreira, L. F. V.; Herance, J. R.; Bourdelande, J. L.; Williams, S. L.; Worrall, D. R.; Insinska-Rak, M.; Sikorski, M. Spectroscopy and photophysics of flavin related compounds: Riboflavin and iso-(6,7)-riboflavin. Chem Phys. 314:239-247; 2005.
[41] Redmond, R. W.; Gamlin, J. N. A compilation of singlet oxygen yields from biologically relevant molecules. Photochem Photobiol. 70:391-475; 1999.
[42] Valko, K. Application of high-performance liquid chromatography based measurements of lipophilicity to model biological distribution. J Chromatogr A. 1037:299-310; 2004.
[43] Livingstone, D. J. Theoretical property predictions. Curr Top Med Chem. 3:1171-1192; 2003.
[44] Oprea, T. I. Current trends in lead discovery: are we looking for the appropriate properties? Mol Divers. 5:199-208; 2002.
[45] Kessel, D.; Luo, Y. Photodynamic therapy: a mitochondrial inducer of apoptosis. Cell Death Differ. 6:28-35; 1999.
[46] Luo, Y.; Chang, C. K.; Kessel, D. Rapid initiation of apoptosis by photodynamic therapy. Photochem Photobiol. 63:528-534; 1996.
[47] Agostinis, P.; Buytaert, E.; Breyssens, H.; Hendrickx, N. Regulatory pathways in photodynamic therapy induced apoptosis. Photochem Photobiol Sci. 3:721-729; 2004.
[48] Niedre, M.; Patterson, M. S.; Wilson, B. C. Direct near-infrared luminescence detection of singlet oxygen generated by photodynamic therapy in cells in vitro and tissues in vivo. Photochem Photobiol. 75:382-391; 2002.
[49] Castano, A. P.; Demidova, T. N.; Hamblin, M. R. Mechanisms in photodynamic therapy: part one—photosensitizers, photochemistry and cellular localization. Photodiag Photodyn Ther. 1:279-293; 2004.
[50] Detty, M. R.; Gibson, S. L.; Wagner, S. J. Current clinical and preclinical photosensitizers for use in photodynamic therapy. J Med Chem. 47:3897-3915; 2004.
[51] Boyle, R. W.; Dolphin, D. Structure and biodistribution relationships of photodynamic sensitizers. Photochem Photobiol. 64:469-485; 1996.
[52] Agostinis, P.; Vantieghem, A.; Merlevede, W.; de Witte, P. A. Hypericin in cancer treatment: more light on the way. Int J Biochem Cell Biol. 34:221-241; 2002.
[53] Wainwright, M.; Crossley, K. B. Methylene Blue—a therapeutic dye for all seasons? J Chemother. 14:431-443; 2002.
[54] Tagmatarchis, N.; Shinohara, H. Fullerenes in medicinal chemistry and their biological applications. Mini Rev Med Chem. 1:339-348; 2001.
[55] Nakamura, E.; Isobe, H. Functionalized fullerenes in water. The first 10 years of their chemistry, biology, and nanoscience. Acc Chem Res. 36:807-815; 2003.
[56] Hasobe, T.; Hattori, S.; Kotani, H.; Ohkubo, K.; Hosomizu, K.; Imahori, H.; Kamat, P. V.; Fukuzumi, S. Photoelectrochemical properties of supramolecular composite of fullerene nanoclusters and 9-mesityl-10-carboxymethylacridinium ion on SnO2. Org Lett. 6:3103-3106; 2004.
[57] Arbogast, J. W.; Foote, C. S.; Kao, M. Electron-transfer to triplet C-60. J Am Chem Soc. 114:2277-2279; 1992.
[58] Guldi, D. M.; Prato, M. Excited-state properties of C(60) fullerene derivatives. Acc Chem Res. 33:695-703; 2000.
[59] Bilski, P.; Belanger, A. G.; Chignell, C. F. Photosensitized oxidation of 2′,7′-dichlorofluorescin: singlet oxygen does not contribute to the formation of fluorescent oxidation product 2′,7′-dichlorofluorescein. Free Radic Biol Med. 33:938-946; 2002.
[60] He, X. Y.; Sikes, R. A.; Thomsen, S.; Chung, L. W.; Jacques, S. L. Photodynamic therapy with photofrin II induces programmed cell death in carcinoma cell lines. Photochem Photobiol. 59:468-473; 1994.
[61] Granville, D. J.; Carthy, C. M.; Jiang, H.; Shore, G. C.; McManus, B. M.; Hunt, D. W. Rapid cytochrome c release, activation of caspases 3, 6, 7 and 8 followed by Bap31 cleavage in HeLa cells treated with photodynamic therapy. FEBS Lett. 437:5-10; 1998.
[62] Gupta, S.; Ahmad, N.; Mukhtar, H. Involvement of nitric oxide during phthalocyanine (Pc4) photodynamic therapy-mediated apoptosis. Cancer Res. 58:1785-1788; 1998.
[63] Kessel, D.; Luo, Y.; Mathieu, P.; Reiners, J. J., Jr. Determinants of the apoptotic response to lysosomal photodamage. Photochem Photobiol. 71:196-200; 2000.
[64] Ben-Dror, S.; Bronshtein, I.; Wiehe, A.; Roder, B.; Senge, M. O.; Ehrenberg, B. On the correlation between hydrophobicity, liposome binding and cellular uptake of porphyrin sensitizers. Photochem Photobiol. 82:695-701; 2006.
[65] Cauchon, N.; Tian, H.; Langlois, R.; La Madeleine, C.; Martin, S.; Ali, H.; Hunting, D.; van Lier, J. E. Structure-photodynamic activity relationships of substituted zinc trisulfophthalocyanines. Bioconjug Chem. 16:80-89; 2005.
[66] Potter, W. R.; Henderson, B. W.; Bellnier, D. A.; Pandey, R. K.; Vaughan, L. A.; Weishaupt, K. R.; Dougherty, T. J. Parabolic quantitative structure-activity relationships and photodynamic therapy: application of a three-compartment model with clearance to the in vivo quantitative structure-activity relationships of a congeneric series of pyropheophorbide derivatives used as photosensitizers for photodynamic therapy. Photochem Photobiol. 70:781-788; 1999.
[67] Ross, M. F.; Da Ros, T.; Blaikie, F. H.; Prime, T. A.; Porteous, C. M.; Severina, II; Skulachev, V. P.; Kjaergaard, H. G.; Smith, R. A.; Murphy, M. P. Accumulation of lipophilic dications by mitochondria and cells. Biochem J. 400:199-208; 2006.
[68] Rottenberg, H. Membrane potential and surface potential in mitochondria: uptake and binding of lipophilic cations. J Membr Biol. 81:127-138; 1984.
[69] Murphy, M. P.; Smith, R. A. Targeting antioxidants to mitochondria by conjugation to lipophilic cations. Annu Rev Pharmacol Toxicol. 47:629-656; 2007.
[70] Petrat, F.; Pindiur, S.; Kirsch, M.; de Groot, H. NAD(P)H, a primary target of 1O2 in mitochondria of intact cells. J Biol Chem. 278:3298-3307; 2003.
[71] Bachowski, G. J.; Ben-Hur, E.; Girotti, A. W. Phthalocyanine-sensitized lipid peroxidation in cell membranes: use of cholesterol and azide as probes of primary photochemistry. J Photochem Photobiol B. 9:307-321; 1991.
[72] Martin, J. P., Jr.; Burch, P. Production of oxygen radicals by photosensitization. Methods Enzymol. 186:635-645; 1990.
[73] Martin, J. P.; Logsdon, N. Oxygen radicals are generated by dye-mediated intracellular photooxidations: a role for superoxide in photodynamic effects. Archives of Biochemistry and Biophysics. 256:39-49; 1987.
[74] Hamblin, M. R.; Hasan, T. Photodynamic therapy: a new antimicrobial approach to infectious disease? Photochem. Photobiol. Sci. 3:436-450; 2004.
[75] Hamblin, M. R.; O'Donnell, D. A.; Murthy, N.; Rajagopalan, K.; Michaud, N.; Sherwood, M. E.; Hasan, T. Polycationic photosensitizer conjugates: effects of chain length and Gram classification on the photodynamic inactivation of bacteria. J Antimicrob Chemother. 49:941-951; 2002.

1. A method for treating a hyperproliferative disorder, comprising:
(a) administering an effective amount of a composition comprising a functionalized fullerene compound to a subject in need thereof, wherein the fullerene compound is a functionized fullerene compound of the formula: 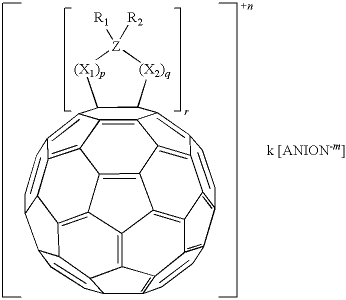
wherein
Z is carbon, nitrogen or phosphorus;
R₁ and R₂ are independently selected from the group consisting of C₁-C₁₂alkyl, C₂-C₁₂alkenyl, C₂-C₁₂alkynyl, C₃-C₈cycloalkyl, (aryl)C₀-C₄alkyl, (heteroaryl)C₀-C₄alkyl, or a group of the formula C(O)—N(R₄)(R₅)(R₆); or
ZR₁R₂ taken in combination form a 3-20 member heterocyclic ring having 1-6 ring heteroatoms selected from nitrogen and phosphorus and having at least one quaternary ammonium cation or quaternary phosphonium cation;
R₄ and R₅ are independently selected from hydrogen or a group selected from C₁-C₁₂alkyl, C₂-C₁₂alkenyl, C₂-C₁₂alkynyl, C₃-C₇cycloalkyl, C₃-C₈ (aryl)C₀-C₄alkyl, and (heteroaryl)C₀-C₄alkyl each of which groups is substituted with 0-3 substituents selected from hydroxy, amino, mono-, di-, or tri-(C₁-C₄alkyl)amino, halogen, quaternary ammonium cations, quaternary phosphonium cations;
R₆ is absent, hydrogen or a group selected from C₁-C₁₂alkyl, C₂-C₁₂alkenyl, C₂-C₁₂alkynyl, C₃-C₇cycloalkyl, C₃-C₈cycloalkyl, (aryl)C₀-C₄alkyl, and (heteroaryl)C₀-C₄alkyl each of which groups is substituted with 0-3 substituents selected from hydroxy, amino, mono-, di-, or tri-(C₁-C₄alkyl)amino, halogen, quaternary ammonium cations, quaternary phosphonium cations;
X₁ and X₂ are independently selected at each occurrence from the group consisting of CH₂ and CHR₃, wherein R₃ is a C₁-C₆alkyl which is independently selected at each occurrence of R₃;
r is 1, 2, 3, or 4;
p and q are independently selected from 0, 1, 2, or 3 such that 0≦(p+q) s≦4;
ANION is at least one organic or inorganic anion;
m is a negative integer corresponding to the net negative charge of each ANION equivalent;
n is a positive integer corresponding to the net positive charge of the substituted buckminsterfullerene cation; and
k is the quotient of n/m;
(b) directing light onto the administered fullerene compound to produce a cytotoxic species; and
(c) inhibiting, blocking, reducing, or decreasing, cell proliferation or cell division in a cell or cells associated with or proximal to the fullerene compound by reaction with the cytotoxic species, thereby treating a hyperproliferative disorder.

2. A method for inducing apoptosis in a cell, comprising:
(a) administering an effective amount of a composition comprising a functionalized fullerene compound to a cell wherein the fullerene compound is a functionized fullerene compound of the formula: 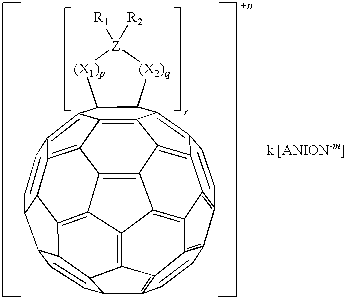
wherein
Z is carbon, nitrogen or phosphorus;
R₁ and R₂ are independently selected from the group consisting of C₁-C₁₂alkyl, C₂-C₁₂alkenyl, C₂-C₁₂alkynyl, C₃-C₈cycloalkyl, (aryl)C₀-C₄alkyl, (heteroaryl)C₀-C₄alkyl, or a group of the formula C(O)—N(R₄)(R₅)(R₆); or
ZR₁R₂ taken in combination form a 3-20 member heterocyclic ring having 1-6 ring heteroatoms selected from nitrogen and phosphorus and having at least one quaternary ammonium cation or quaternary phosphonium cation;
R₄ and R₅ are independently selected from hydrogen or a group selected from C₁-C₁₂alkyl, C₂-C₁₂alkenyl, C₂-C₁₂alkynyl, C₃-C₇cycloalkyl, C₃-C₈ (aryl)C₀-C₄alkyl, and (heteroaryl)C₀-C₄alkyl each of which groups is substituted with 0-3 substituents selected from hydroxy, amino, mono-, di-, or tri-(C₁-C₄alkyl)amino, halogen, quaternary ammonium cations, quaternary phosphonium cations;
R₆ is absent, hydrogen or a group selected from C₁-C₁₂alkyl, C₂-C₁₂alkenyl, C₂-C₁₂alkynyl, C₃-C₇cycloalkyl, C₃-C₈cycloalkyl, (aryl)C₀-C₄alkyl, and (heteroaryl)C₀-C₄alkyl each of which groups is substituted with 0-3 substituents selected from hydroxy, amino, mono-, di-, or tri-(C₁-C₄alkyl)amino, halogen, quaternary ammonium cations, quaternary phosphonium cations;
X₁ and X₂ are independently selected at each occurrence from the group consisting of CH₂ and CHR₃, wherein R₃ is a C₁-C₆alkyl which is independently selected at each occurrence of R₃;
r is 1, 2, 3, or 4;
p and q are independently selected from 0, 1, 2, or 3 such that 0≦(p+q)≦4;
ANION is at least one organic or inorganic anion;
m is a negative integer corresponding to the net negative charge of each ANION equivalent;
n is a positive integer corresponding to the net positive charge of the substituted buckminsterfullerene cation; and
k is the quotient of n/m;
(b) directing light onto the administered fullerene compound to produce a cytotoxic species; and
(c) inducing apoptosis in the cell associated with or proximal to the fullerene compound by reaction with the cytotoxic species.

3. A method for treating cancer, comprising:
(a) administering an effective amount of a composition comprising a functionalized fullerene compound to a subject in need thereof, wherein the fullerene compound is a functionized fullerene compound of the formula: 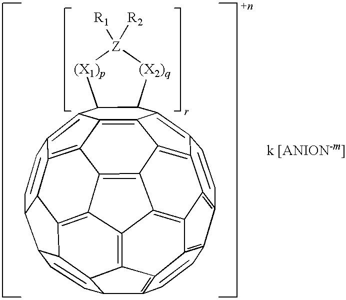
wherein
Z is carbon, nitrogen or phosphorus;
R₁ and R₂ are independently selected from the group consisting of C₁-C₁₂alkyl, C₂-C₁₂alkenyl, C₂-C₁₂alkynyl, C₃-C₈cycloalkyl, (aryl)C₀-C₄alkyl, (heteroaryl)C₀-C₄alkyl, or a group of the formula C(O)—N(R₄)(R₅)(R₆); or
ZR₁R₂ taken in combination form a 3-20 member heterocyclic ring having 1-6 ring heteroatoms selected from nitrogen and phosphorus and having at least one quaternary ammonium cation or quaternary phosphonium cation;
R₄ and R₅ are independently selected from hydrogen or a group selected from C₁-C₁₂alkyl, C₂-C₁₂alkenyl, C₂-C₁₂alkynyl, C₃-C₇cycloalkyl, C₃-C₈ (aryl)C₀-C₄alkyl, and (heteroaryl)C₀-C₄alkyl each of which groups is substituted with 0-3 substituents selected from hydroxy, amino, mono-, di-, or tri-(C₁-C₄alkyl)amino, halogen, quaternary ammonium cations, quaternary phosphonium cations;
R₆ is absent, hydrogen or a group selected from C₁-C₁₂alkyl, C₂-C₁₂alkenyl, C₂-C₁₂alkynyl, C₃-C₇cycloalkyl, C₃-C₈cycloalkyl, (aryl)C₀-C₄alkyl, and (heteroaryl)C₀-C₄alkyl each of which groups is substituted with 0-3 substituents selected from hydroxy, amino, mono-, di-, or tri-(C₁-C₄alkyl)amino, halogen, quaternary ammonium cations, quaternary phosphonium cations;
X₁ and X₂ are independently selected at each occurrence from the group consisting of CH₂ and CHR₃, wherein R₃ is a C₁-C₈alkyl which is independently selected at each occurrence of R₃;
r is 1, 2, 3, or 4;
p and q are independently selected from 0, 1, 2, or 3 such that 0≦(p+q)≦4;
ANION is at least one organic or inorganic anion;
m is a negative integer corresponding to the net negative charge of each ANION equivalent;
n is a positive integer corresponding to the net positive charge of the substituted buckminsterfullerene cation; and
k is the quotient of n/m;
(b) directing light onto the administered fullerene compound to produce a cytotoxic species; and
(c) killing a cell or cells in said subject associated with or proximal to the fullerene compound by reaction with the cytotoxic species, thereby treating a cancer.

4. The method of claim 1, wherein the fullerene is a compound of the formula 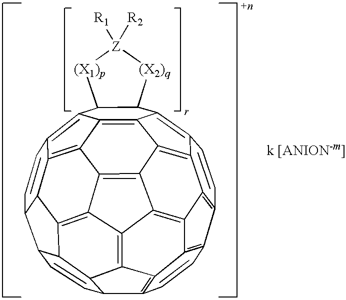
wherein
Z is nitrogen or phosphorus;
X₁ and X₂ are methylene;
p=q=1;
R₁ and R₂ are independently selected C₁-C₆alkyl, (aryl)C₀-C₁alkyl, or (heteroaryl)C₀-C₁alkyl;
r is 2, 3, or 4; and
n≧r.

5. The method of claim 1, wherein the fullerene is a compound of the formula 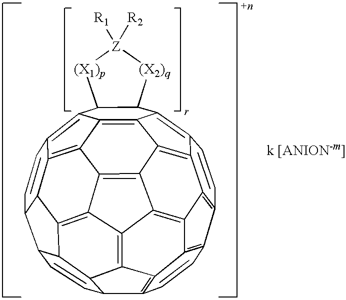
wherein
Z is nitrogen or phosphorus;
X₁ and X₂ are methylene;
p=q=1;
R₁ is C₁-C₆alkyl, (aryl)C₀-C₁alkyl, or (heteroaryl)C₀-C₁alkyl;
R₂ is (aryl)C₀-C₁alkyl, or (heteroaryl)C₀-C₁alkyl;
r is 1, 2, 3, or 4; and
n≧r.

6. The method of claim 1, wherein the fullerene is a compound of the formula 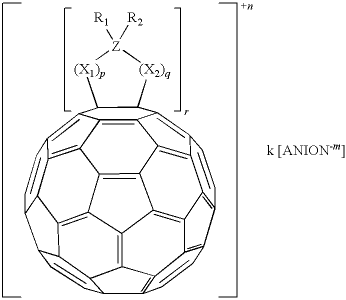
wherein
Z is nitrogen;
X₁ and X₂ are methylene;
p=q=1;
R₁ and R₂ are independently selected from methyl, ethyl, propyl or isopropyl;
r is 2, 3, or 4; and
n≧r.

7. The method of claim 1, wherein the fullerene is a compound of the formula 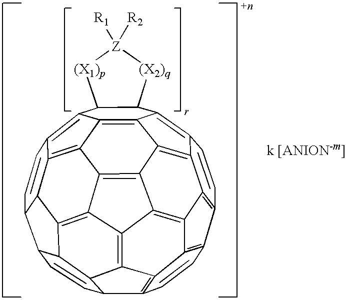
Z is carbon;
p=q=0;
R₁ and R₂ are independently selected groups of the formula C(O)—N(R₄)(R₅)(R₆); or
ZR₁R₂ taken in combination form a 6-20 member heterocyclic ring having 1-6 ring heteroatoms selected from nitrogen and phosphorus and having at least one quaternary ammonium cation or quaternary phosphonium cation;
R₄ and R₅ are independently selected from hydrogen or a group selected from C₁-C₁₂alkyl, C₂-C₁₂alkenyl, C₂-C₁₂alkynyl, C₃-C₇cycloalkyl, C₃-C₈ (aryl)C₀-C₄alkyl, and (heteroaryl)C₀-C₄alkyl each of which groups is substituted with 0-3 substituents selected from hydroxy, amino, di-, or tri-(C₁-C₂alkyl)amino, halogen, quaternary ammonium cations, quaternary phosphonium cations; and
R₆ is absent, hydrogen or a group selected from C₁-C₁₂alkyl, C₂-C₁₂alkenyl, C₂-C₁₂alkynyl, C₃-C₇cycloalkyl, C₃-C₈cycloalkyl, (aryl)C₀-C₄alkyl, and (heteroaryl)C₀-C₄alkyl each of which groups is substituted with 0-3 substituents selected from hydroxy, amino, mono-, di-, or tri-(C₁-C₂alkyl)amino, halogen, quaternary ammonium cations, quaternary phosphonium cations.

8. The method of claim 7, wherein the fullerene is a compound of the formula 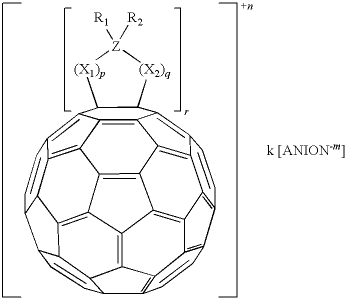
wherein
R₁ and R₂ are independently selected groups of the formula C(O)—N(R₄)(R₅)(R₆);
R₄ is C₂-C₆alkyl substituted with 1-3 substitutents selected from hydroxy, amino, di-, or tri-(C₁-C₂alkyl)amino, and quaternary ammonium cations;
R₅ is hydrogen, C₁-C₆alkyl substituted with 0-3 substitutents selected from hydroxy, amino, and quaternary ammonium cations; and
R₆ is absent, hydrogen, or C₁-C₆alkyl substituted with 0-3 substitutents selected from hydroxy, amino, di-, or tri-(C₁-C₂alkyl)amino, and quaternary ammonium cations.

9. The method of claim 7, wherein the fullerene is a compound of the formula 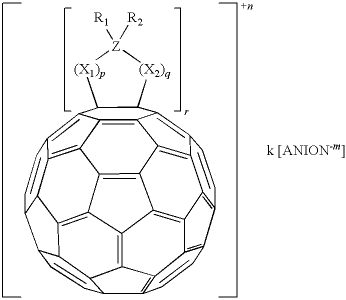
wherein
R₁ and R₂ are the same and are selected from the group consisting of: 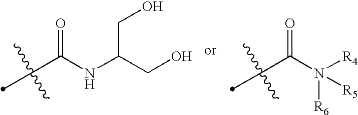
wherein R₄ is methyl, ethyl or propyl or isopropyl;
R₅ and R₆ are independently selected from methyl, ethyl, 2-(N,N-dimethylamino)ethyl, 3-(N,N-dimethylamino)propyl, 2-(N,N,N-trimethylammonium)ethyl, or 3-(N,N,N-trimethylammonium)propyl.

10. The method of claim 9, wherein the fullerene is a compound of the formula 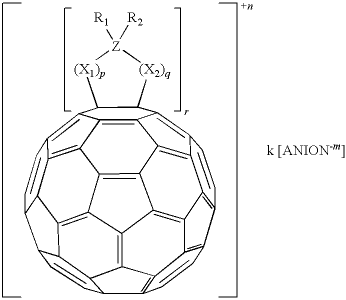
wherein
r is 1.

11. The method of claim 9, wherein the fullerene is a compound of the formula 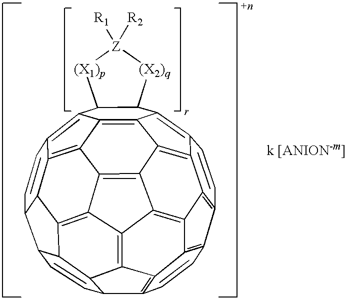
wherein
r is 2.

12. The method of claim 9, wherein the fullerene is a compound of the formula 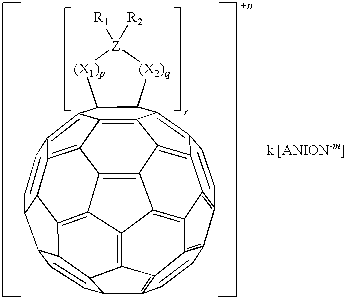
wherein
r is 3.

13. The method of claim 1, wherein the fullerene is a compound of the formula 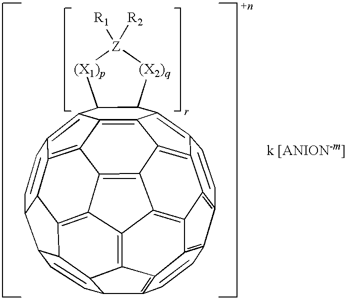
wherein
p=q=0; and
ZR₁R₂, taken in combination, form a 7-20 member heterocyclic ring having 2 to 6 nitrogen atoms wherein at least one of the nitrogen atoms is a quaternary ammonium cation.

14. The method of claim 13, wherein the fullerene is a compound of the formula 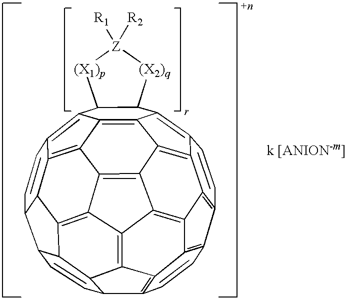
wherein
ZR₁R₂ is a heterocyclic ring of the formula: 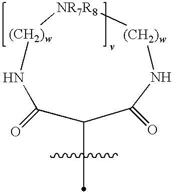
wherein
w is independently selected at each occurrence from 1, 2 or 3;
v is 0, 1, 2, or 3;
R₇ is independently selected at each occurrence from hydrogen, C₁-C₆alkyl substituted with 0-3 substitutents selected from hydroxy, amino, and quaternary ammonium cations; and
R₈ is independently selected at each occurrence from absent, hydrogen, or C₁-C₆alkyl substituted with 0-3 substitutents selected from hydroxy, amino, di-, or tri-(C₁-C₂alkyl)amino, and quaternary ammonium cations; and wherein at least one NR₇R₈ is a quaternary ammonium cation or is substituted by a quaternary ammonium cation.

15. The method of claim 14, wherein the fullerene is a compound of the formula 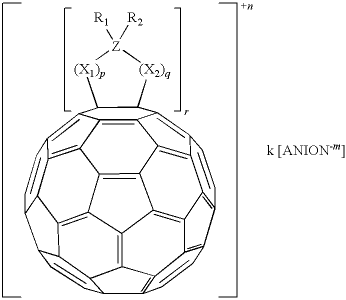
wherein
ZR₁R₂ is a heterocyclic ring of the formula: 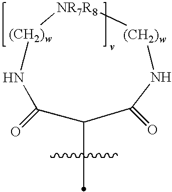
and wherein
v is 1, 2 or 3;
w is 2;
R₇ is independently selected from the group of methyl, ethyl or propyl or isopropyl; and
R₈ are independently selected from methyl, ethyl, 2-(N,N-dimethylamino)ethyl, 3-(N,N-dimethylamino)propyl, 2-(N,N,N-trimethylammonium)ethyl, or 3-(N,N,N-trimethylammonium)propyl.

16. The method of claim 1, wherein the fullerene is functionalized with a cationic organic moiety.

17. The method of claim 16, wherein the fullerene is functionalized with a nonionic organic moiety.

18. The method of claim 16, further comprising:
washing away excess fullerenes that are not associated with the cell or cells to be treated prior to the step of directing light onto the associated fullerene compound.

19. The method of claim 16, wherein the composition is applied as a solution having a fullerene concentration of between 1 and 100 micromolar.

20. The method of claim 16, wherein the light is visible light is provided at an intensity of 0.5 and 160 Joules per square centimeter.

21. The method of claim 16, wherein visible light is provided at an intensity between 0.5 and 20 Joules per square centimeter.

22. The method of claim 16, wherein the functionalized fullerene is water soluble.

23. The method of claim 3, wherein the killing is selective for the cancer and tumor cells of the subject.

24. The method of claim 3, wherein the cancer is a cancer of the breast, respiratory tract, brain, reproductive organs, digestive tract, urinary tract, eye, liver, skin, head and neck, thyroid, parathyroid or a distant metastasis of a solid tumor.

25. The method of claim 3, wherein the cancer is a lymphoma, sarcoma, melanoma or leukemia.

26. The method of claim 16, wherein the method further comprises administering at least one further active compound.

27. The method of claim 26, wherein the further active compound is an anti-hyperproliferative agent.
